(19) United States
(12) Patent Application Publication
(10) Pub. No.: US 2018/0168574 A1
(43) Pub. Date: Jun. 21, 2018

(54) CONVERTIBLE SURGICAL TISSUE STAPLERS AND APPLICATIONS USING THEREOF

(71) Applicant: STEPWISE LTD, Tel-Aviv (IL)

(72) Inventors: Simon ROBINSON, Ein Sarid (IL); Inbal LOITNER, Rishon Lezion (IL); Yehuda BACHAR, Givaat Shmuel (IL); Ronny WINSHTEIN, Ramat Hasharon (IL)

(21) Appl. No.: 15/127,508

(22) PCT Filed: Mar. 18, 2015
(86) PCT No.: PCT/IB2015/051981

§ 371 Date: Sep. 20, 2016

Related U.S. Application Data
(60) Provisional application No. 61/968,265, filed on Mar. 20, 2014.

(51) Int. Cl. A61B 17/072; A61B 17/064

(52) U.S. Cl. CPC A61B 17/072; A61B 2017/00477; A61B 17/064

(57) ABSTRACT

Convertible surgical tissue staplers and applications thereof, having small sizes and high suitability for introduction and delivery through small surgical incisions or cuts, natural orifices, or laparoscopic ports, followed by deployment to full operational configurations. Convertible surgical tissue stapler may be a linear tissue stapler such as endo cutter stapler. Linear tissue stapler includes: elongated body having longitudinal axis; and stapling head, including first and second members, at least one with rows of tissue fasteners. Convertible from delivery configuration, whereby members are consecutively arranged lengthwise, with overall diameter smaller than minimal inner diameter, to deployment configuration, whereby members are juxtapositionally arranged, with overall diameter equal to or greater than minimal inner diameter. Also disclosed are methods of surgically fastening or/and cutting tissue inside a body, for example, using a linear tissue stapler, and of deploying a linear tissue stapler to operational form.

### RELATED APPLICATION

This application claims the benefit of priority under 35 USC 119(e) of U.S. Provisional Patent Application No. 61/968,265, filed Mar. 20, 2014, entitled “CONVERTIBLE SURGICAL STAPLERS AND METHODS”, and of U.S. Provisional Patent Application No. 62/050,476, filed Sep. 15, 2014, entitled “ENDOSCOPIC CUTTER STAPLERS AND METHODS”, the contents of which are incorporated herein by reference in their entirety.

### FIELD OF THE INVENTION

The present invention, in some embodiments thereof, relates to surgical types of tissue fastener (staple) applicators and cutters, and in particular, to surgical linear tissue staplers and endo cutter staplers, and applications using thereof, for example, for surgically fastening (stapling) or/and cutting tissue inside a body.

### BACKGROUND OF THE INVENTION

There is an on-going trend towards designing, and using, surgical tools with smallest possible diameters, while maintaining, and possibly also improving, functionality of surgical practices, particularly, as relating to minimally invasive surgical procedures. In cases of minimally invasively introduced laparoscopic tools, smaller diameter may contribute to smaller incisions or cuts made to the skin and soft tissues underneath. In cases of natural orifice (e.g., orally) introduction of endoscopic tools, smaller diameter may contribute to the possibility or/and ease of adding additional tools or other, larger, tools in parallel, via the natural orifice.

The trend of using smaller diameter surgical tools also encompasses design efforts of surgical staplers, including, for example, linear staplers and endo (endoscopic) cutter staplers. Surgical staplers are commonly introduced through laparoscopic ports having diameters of at least about 12 mm, indicating a need for surgical staplers to have smaller designs capable of being introduced through natural orifices or through laparoscopic ports having diameters of about 6 mm or less.

Some teachings in the art include focusing on new configurations of surgical staples and ways of applying such staples (e.g., attaching staples to and through tissue walls), which may enable using staplers having smaller overall sizes. Examples of such teachings are disclosed in U.S. Pat. Nos. 8,556,935; 8,403,956; and 8,365,973, the disclosures of which are fully incorporated herein by reference.

### SUMMARY OF THE INVENTION

The present invention, in some embodiments thereof, relates to surgical types of tissue fastener (staple) applicators and cutters, and in particular, to convertible surgical linear tissue staplers and endo cutter staplers, and applications using thereof, for example, for surgically fastening (stapling) or/and cutting tissue inside a body.

Some embodiments of the present invention are of surgical tissue staplers and endo cutter staplers having (structural and functional) features relating to their being relatively small in size and highly suitable for efficient introduction and delivery through small surgical incisions or cuts, or, through natural orifices or laparoscopic ports, followed by deployment to full operational configurations. For example, according to an aspect of some embodiments of the invention, there is provided a method of deploying a linear tissue stapler to an operational form. These and other features of some embodiments of the present invention may be advantageous in the field and art of the invention, particularly, compared to existing configurations and sizes of surgical tissue fastener (stapler) applicators and cutters, and methods of using them.

According to an aspect of some embodiments of the present invention, there is provided a linear tissue stapler, comprising: an elongated body having a longitudinal axis; and a stapling head, including at least a first member and a second member, at least one of the first member and the second member includes at least one row of tissue fasteners; wherein the linear tissue stapler is convertible from a delivery configuration, whereby the elongated body and the stapling head are passable through a passage enclosing a minimal inner diameter, to a deployment configuration, whereby the stapling head is operational; wherein, in the delivery configuration, the first member and the second member are consecutively arranged lengthwise relative to the longitudinal axis, such that an overall diameter thereof is smaller than the minimal inner diameter, and, in the deployment configuration, the first member and the second member are juxtapositionally arranged relative to the longitudinal axis, such that an overall diameter thereof is equal to or greater than the minimal inner diameter.

According to some embodiments of the invention, the linear tissue stapler comprises a first track, wherein, in the delivery configuration, the first member contacts the first track at a first location, and, in the deployment configuration, the first member contacts the first track at a second location.

According to some embodiments of the invention, the linear tissue stapler comprises a second track, wherein, in the delivery configuration, the second member contacts the second track at a third location, and, in the deployment configuration, the second member contacts the second track at a fourth location.

According to some embodiments of the invention, the first track is located within a lumen of the elongated body, and the second track is located within the lumen of the elongated body.

According to some embodiments of the invention, upon converting from the delivery configuration to the deployment configuration, the first track is configured such that the first member travels lengthwise or/and rotates relative to the longitudinal axis.

According to some embodiments of the invention, upon converting from the delivery configuration to the deployment configuration, the second track is configured such that the second member travels lengthwise or/and rotates relative to the longitudinal axis.

According to some embodiments of the invention, the minimal inner diameter is equal to or less than about 12 mm, optionally, equal to or less than about 6 mm.

According to some embodiments of the invention, the at least one of the first member and the second member includes at least three of the rows of the tissue fasteners, optionally, at least five of the tissue fasteners.

According to some embodiments of the invention, the first member is a first tissue fastener applying member, and the second member is a second tissue fastener applying member.

According to some embodiments of the invention, in the deployment configuration, the first tissue fastener applying member and the second tissue fastener applying member are interlocked with each other to rigidly form a single stapling jaw.

According to some embodiments of the invention, the linear tissue stapler further comprising an anvil jaw, wherein, in the deployment configuration, the anvil jaw and the stapling jaw oppose each other and are movable relative to each other, from an open position, in which the jaws are spaced apart, to a closed position, in which the jaws are in close proximity to each other.

According to some embodiments of the invention, the second member is an anvil jaw, and the first member is a stapling jaw including a plurality of the rows of tissue fasteners, the rows being parallel to each other.

According to some embodiments of the invention, the stapling head includes a stapling head actuating assembly, and wherein the anvil jaw is pivotally connected to the elongated body and is operable with the stapling head actuating assembly.

According to some embodiments of the invention, the anvil jaw includes a plurality of depressions oppositely positioned relative to a corresponding number of the tissue fasteners, such that upon clamping the anvil jaw against the stapling jaw, each of the fasteners is forcibly bent to a closed shape against a corresponding one of the depressions.

According to some embodiments of the invention, the anvil jaw includes convertible anvil members having a first position, in the delivery configuration, with an overall width thereof being equal to or smaller than the minimal inner diameter, and having a second position, in the deployment configuration, with the overall width thereof exceeding the minimal inner diameter.

According to some embodiments of the invention, the linear tissue stapler further comprises a linear fasteners cartridge housing a plurality of the tissue fasteners.

According to some embodiments of the invention, the linear fasteners cartridge is replaceable or/and ejectable from the stapling head.

According to some embodiments of the invention, the linear tissue stapler further comprises a driving member including a number of runners, each of the runners is slidable along a corresponding grooved route extending along a length of one of the first and second members.

According to some embodiments of the invention, the at least one row of the tissue fasteners includes a number of grooved slots, each of the grooved slots houses a staple pusher having a cam head, such that upon sliding the runners distally along the grooved route, each of the staple pushers forcibly moves vertically in a corresponding one of the grooved slots, whereby the corresponding tissue fasteners are pressed towards corresponding depressions oppositely positioned relative to the tissue fasteners.

According to some embodiments of the invention, in the deployment configuration, a first driving member and a second driving member are interlocked with each other to form a single driver.

According to some embodiments of the invention, the driver includes, or is connected to, a top plate pressing against a surface area of one of the first and second members, the top plate is interconnected to a bottom plate pressing against an opposing surface area, wherein the top plate and the bottom plate are elastically connected or fixedly distanced with respect to each other such that, at any driver location along the stapling head, via actuating the driver to move distally or proximally, the opposing surface areas are forced to approximate each other at the driver location.

According to some embodiments of the invention, via the actuating of the driver, at least one of the tissue fasteners is pressed between the opposing areas, towards a corresponding depression oppositely positioned relative to the tissue fastener.

According to some embodiments of the invention, the linear tissue stapler comprises a driving clutch member engageable with both of the first and second driving members, wherein, in the delivery configuration, the driving clutch member is disengaged from the driving members, and, in the deployment configuration, the driving clutch member engages with the driving members.

According to some embodiments of the invention, the driving clutch member is configured to drive lengthwise along the longitudinal axis in a spaced passage extending along the stapling head, when in the deployment configuration.

According to some embodiments of the invention, the driving clutch member includes, or is connected to, a blade having a sharp edge, the sharp edge is configured to cut through body tissue clasped by the stapling head in response to distal sliding of the driving clutch member along the spaced passage.

According to some embodiments of the invention, in the delivery configuration, the driving clutch member is positioned between the elongated body and the stapling head.

According to some embodiments of the invention, in the delivery configuration, the driving clutch member is positioned proximally to the stapling head.

According to some embodiments of the invention, the first tissue fastener applying member is hingedly connected to the second tissue fastener applying member, such that the second tissue fastener applying member is rotatable with respect to the first tissue fastener applying member, wherein, in the delivery configuration, a second end of the second tissue fastener applying member is positioned distally to the distal end of the first tissue fastener applying member, and, in the deployment configuration, the second end of the second tissue fastener applying member is positioned proximally to the distal end of the first tissue fastener applying member.

According to some embodiments of the invention, the first member transversally shifts between the delivery configuration and the deployment configuration, to a position juxtaposed to the second member.

According to an aspect of some embodiments of the present invention, there is provided a method of surgically fastening or/and cutting tissue inside a body, the method comprising: providing a passage connecting between environment outside of the body and a location inside the body in a vicinity of the tissue; passing a linear tissue stapler through the passage towards the inside body location, the linear tissue stapler comprises an elongated body having a longitudinal axis, and, a stapling head including a first member and a second member arranged in a delivery configuration, in which the first and second members are consecutively arranged lengthwise relative to the longitudinal axis; effecting emergence of the first member and the second member out of the passage; converting the linear tissue stapler from the delivery configuration to a deployment configuration, in which the first member and the second member are juxtapositionally arranged to relative to the longitudinal axis and interlocked with each other to rigidly form an operational stapling head comprising a stapling jaw and an anvil jaw; clasping the tissue between the stapling jaw and the anvil jaw; and actuating the stapling head to release a plurality of tissue fasteners from the stapling jaw through the tissue towards the anvil jaw.

According to some embodiments of the invention, the method further comprises providing a blade in a spaced passage extending along the stapling jaw of the stapling head, wherein the actuating includes, or is followed by, sliding the blade along the spaced passage so as to cut through the clasped tissue with a sharp edge of the blade.

According to some embodiments of the invention, the passage encloses a minimal inner diameter equal to or less than about 6 mm, wherein the stapling jaw, in the deployment configuration, has overall diameter greater than the minimal inner diameter.

According to some embodiments of the invention, the linear tissue stapler further comprises a plurality of driving members, each the driving member includes a number of runners, wherein each of the runners is slidable along a corresponding grooved route extending along a length of the first member and/or the second member.

According to some embodiments of the invention, the converting includes connecting the driving members to form a single driver.

According to some embodiments of the invention, the method comprises: providing a driving clutch member proximally to the elongated body; engaging the driving clutch member with the driving members; and driving the driving clutch member lengthwise along the longitudinal axis in the spaced passage extending along the stapling head.

According to an aspect of some embodiments of the present invention, there is provided a method of deploying a linear tissue stapler to an operational form, the method comprising: providing a linear tissue stapler comprising an elongated body having a longitudinal axis, and a stapling head including a first member and a second member consecutively arranged lengthwise relative to the longitudinal axis; and repositioning at lease the first member or/and the second member such that the first member and the second member are juxtapositionally arranged relative to the longitudinal axis, whereby the stapling head becomes operational with a stapling jaw hingedly connected to an anvil jaw and opposing thereto.

According to some embodiments of the invention, the first member is a first tissue fastener applying member and the second member is a second tissue fastener applying member, the method further comprising: interlocking the first tissue fastener applying member and the second tissue fastener applying member with each other to rigidly form the stapling jaw configured to move with respect to the anvil jaw from an open position, in which the jaws are spaced apart, to a closed position, in which the jaws are in close proximity to each other.

According to some embodiments of the invention, the first member is the stapling jaw and the second member is the anvil jaw.

According to some embodiments of the invention, the repositioning increases diameter of a circumscribed circle around a maximal transverse cross section of the linear tissue stapler.

According to some embodiments of the invention, the diameter of the circumscribed circle increases in magnitude from less than or equal to about 6 mm, to greater than about 6 mm.

According to some embodiments of the invention, the repositioning includes distally shifting the first member or/and the second member to proximity of the elongated body.

According to some embodiments of the invention, the repositioning includes rotating the first member or/and the second member.

According to some embodiments of the invention, the first tissue fastener applying member is provided with a first driving member and the second tissue fastener applying member is provided with a second driving member, and wherein the interlocking includes connecting the first driving member and the second driving member to form a single driver.

According to some embodiments of the invention, the method comprises: providing a driving clutch member proximally to the elongated body; and engaging the driving clutch member with the driver.

According to an aspect of some embodiments of the present invention, there is provided a linear tissue stapler, comprising: a stapling jaw, including parallel rows of tissue fasteners; and an anvil jaw; wherein the linear tissue stapler is convertible from a delivery configuration, whereby the stapling jaw and the anvil jaw are passable through a passage enclosing a minimal inner diameter, to a deployment configuration including an operational stapling head; wherein, in the delivery configuration, the stapling jaw and the anvil jaw are consecutively arranged lengthwise such that a maximal stapler cross-sectional dimension is smaller than the minimal inner diameter, and in the deployment configuration, the stapling jaw and the anvil jaw are juxtapositionally arranged and form the operational stapling head, whereby the maximal stapler cross-sectional dimension is equal to or greater than the minimal inner diameter.

According to some embodiments of the invention, the linear tissue stapler further comprises an elongated body including an elongated body distal end.

According to some embodiments of the invention, in the delivery configuration, the anvil jaw is at least partly positioned proximally to the elongated body distal end, and in the deployment configuration, the anvil jaw is at least partly positioned distally to the elongated body distal end.

According to some embodiments of the invention, the stapling jaw is coupled to the elongated body distal end and lying distally thereto in both of the delivery configuration and the deployment configuration.

According to some embodiments of the invention, the anvil jaw interlocks with the elongated body distal end or/and with the stapling jaw in the deployment configuration to form the operational stapling head.

According to some embodiments of the invention, in the delivery configuration, the anvil jaw is at least partly positioned distally to the stapling jaw.

According to some embodiments of the invention, the stapling jaw is coupled to the elongated body distal end and lying distally thereto in both of the delivery configuration and the deployment configuration.

According to some embodiments of the invention, the anvil jaw interlocks with the stapling jaw in the deployment configuration, to form the operational stapling head distally adjacent the elongated body distal end.

According to some embodiments of the invention, when in the deployment configuration, the anvil jaw overlaps the stapling jaw.

According to some embodiments of the invention, when in the deployment configuration, and when forming the operational stapling head, the stapling jaw and the anvil jaw are movable with respect to each other from an open position, in which the jaws are spaced apart, to a closed position, in which the jaws are in close proximity to each other.

According to some embodiments of the invention, at the converting from the delivery configuration to the deployment configuration, the anvil jaw slides proximally or distally.

According to some embodiments of the invention, the at the converting from the delivery configuration to the deployment configuration, the anvil jaw swivels towards the stapling jaw.

According to some embodiments of the invention, the anvil jaw is configured to revolve around a point located thereon.

According to some embodiments of the invention, the anvil jaw configured to translate relative to the stapling jaw.

According to some embodiments of the invention, the minimal inner diameter is equal to or less than about 8 mm.

According to some embodiments of the invention, in the deployment configuration, the maximal stapler cross-sectional dimension is equal to or greater than about 5 mm.

According to some embodiments of the invention, the stapling jaw includes at least two rows of tissue fasteners.

According to some embodiments of the invention, the at least one of the tissue fasteners is a surgical staple.

According to some embodiments of the invention, the linear tissue stapler further comprises a driver including a blade having a sharp edge extending between the jaws, and configured to travel along a spaced passage between separated opposing portions of the stapling head.

All technical or/and scientific words, terms, or/and phrases, used herein have the same or similar meaning as commonly understood by one of ordinary skill in the art to which the invention pertains, unless otherwise specifically defined or stated herein. Methods, materials, and examples described herein are illustrative only and are not intended to be necessarily limiting. Although methods or/and materials equivalent or similar to those described herein can be used in practicing or/and testing embodiments of the invention, exemplary methods or/and materials are described below. In case of conflict, the patent specification, including definitions, will control.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1A schematically illustrates an isometric view of an exemplary embodiment of a cutting stapler, in accordance with some embodiments of the invention;

FIG. 1B schematically illustrates an enlarged partial view of an exemplary embodiment of a stapling jaw, in accordance with some embodiments of the invention;

FIGS. 2A-2B schematically illustrate exemplary embodiments of a delivery configuration and a deployment configuration, respectively, of a first exemplary embodiment of a linear tissue stapler, in accordance with some embodiments of the invention;

FIGS. 3A-3B schematically illustrate exemplary embodiments of a delivery configuration and a deployment configuration, respectively, of a second exemplary embodiment of a linear tissue stapler, in accordance with some embodiments of the invention;

FIGS. 4A-4B schematically illustrate exemplary embodiments of a delivery configuration and a deployment configuration, respectively, of a third exemplary embodiment of a linear tissue stapler, in accordance with some embodiments of the invention:

FIGS. 5A-5B schematically illustrate front view of exemplary embodiments of a delivery configuration and a deployment configuration, respectively, of a fourth exemplary embodiment linear tissue stapler, in accordance with some embodiments of the invention;

FIG. 6 schematically illustrates an exemplary embodiment of a track for forcing certain maneuverability of a tissue fastener applying member between a delivery configuration and a deployment configuration, in accordance with some embodiments of the invention;

FIG. 7 schematically illustrates an exemplary embodiment of a tissue fastener applying member configured for deployment with a replaceable staples cartridge, in accordance with some embodiments of the invention;

FIG. 8 schematically illustrates a transverse cut view of an exemplary embodiment of a stapling head of an exemplary convertible linear stapler, in accordance with some embodiments of the invention;

FIGS. 9A-9C schematically illustrate exemplary embodiments of components of the exemplary stapling head shown in FIG. 8, in accordance with some embodiments of the invention;

FIGS. 10A-10H schematically illustrate exemplary embodiments of different scenarios representing possible exemplary steps in a method of deploying a convertible linear tissue stapler and surgically affecting a tissue in a body, in accordance with some embodiments of the invention;

FIGS. 11A-11B schematically illustrate exemplary embodiments of a delivery configuration and a deployment configuration, respectively, in side views of an exemplary embodiment of a linear tissue stapler, in accordance with some embodiments of the invention;

FIGS. 12A-12C schematically illustrate side views of exemplary embodiments of different shifting mechanisms between a delivery configuration and a deployment configuration of exemplary embodiment of linear tissue staplers, in accordance with some embodiments of the invention;

FIGS. 13A-13E schematically illustrate side views of exemplary embodiments of different positions in deploying an exemplary embodiment of a linear tissue stapler configured to have its anvil jaw positioned distally to the stapling jaw when in the delivery configuration, in accordance with some embodiments of the invention; and

FIGS. 14A-14C illustrate isometric views of exemplary embodiments of different positions in deploying an exemplary embodiment of a linear tissue stapler configured to have its anvil jaw positioned proximally to the stapling jaw when in the delivery configuration, in accordance with some embodiments of the invention.

### DESCRIPTION OF SPECIFIC EMBODIMENTS OF THE INVENTION

The present invention, in some embodiments thereof, relates to surgical types of tissue fastener (staple) applicators and cutters, and in particular, to convertible surgical linear tissue staplers and endo cutter staplers, and applications using thereof, for example, for fastening (stapling) or/and cutting tissue inside a body.

Some embodiments of the present invention are of surgical tissue staplers and endo cutter staplers having (structural and functional) features relating to their being relatively small in size and highly suitable for efficient introduction and delivery through small surgical incisions or cuts, or, through natural orifices or laparoscopic ports, followed by deployment to full operational configurations. For example, according to an aspect of some embodiments of the invention, there is provided a method of deploying a linear tissue stapler to an operational form. These and other features of some embodiments of the present invention may be advantageous in the field and art of the invention, particularly, compared to existing configurations and sizes of surgical tissue fastener (stapler) applicators and cutters, and methods of using them.

Some embodiments of the present invention may be described in the context of exemplary laparoscopic surgical procedures for ease of description and understanding. However, implementation and practice of the invention are not limited to the specifically described exemplary embodiments of devices and methods, and may be adapted to various clinical applications without departing from the overall scope of the invention. For example, some embodiments of the devices and related methods, and associated features and concepts thereof, described herein, may be used for other surgical procedures and applications, such as, but not limited to: single-port laparoscopy, endoscopy, and NOTES (‘Natural Orifice Translumenal Endoscopic Surgery’) assisted endoscopic or laparoscopic surgeries.

The term ‘surgical stapler’, as used herein, refers to a medical device which is used to place surgical fasteners, such as in a form of staples. The terms ‘surgical fastener’ or ‘tissue fastener’, as used herein, refer to any pronged artifact that can pierce through tissue layers for fastening, securing, fixating, or bonding, them together. A surgical fastener may include one leg or a plurality of legs; each leg is pronged at its end and sized to pass through a plurality of soft tissue layers. A surgical fastener may be elastic in order to bounce from a stressed configuration (e.g., by stretching or compressing its legs opened or closed) to a decreased stressed configuration during deployment, may be rigid enough in order to preclude deformation during deployment, or may be plastically deformable during deployment, such as in the case of surgical tissue staples, where the legs are forced to bend to a fixedly deformed closed configuration, and are commonly used to close wounds or openings in place of sutures.

Surgical staplers may be designed for open surgeries, or, for endoscopic or laparoscopic surgeries, may or may not include tissue cutting means, and may be disposable or reusable. Surgical staplers are optionally loaded with disposable cartridges containing fasteners (e.g., staples). The stapler line may be straight, curved, or circular.

A surgical stapler, as other types of staplers, includes two opposing jaws pivotally movable one with the other: a ‘stapling jaw’, which houses the cartridge/staples and optionally includes means to push the staples to protrude towards the opposing jaw; and an ‘anvil jaw’, having a number of recesses or grooves corresponding in number and position to the staples in ‘stapling jaw’. Upon grasping and compressing a tissue with the jaws, each staple penetrates through the tissue with each of its two pronged legs which are then inwardly bent as they press against the corresponding anvil recesses.

The terms ‘linear tissue stapler’, ‘linear stapler’ and ‘surgical linear stapler’, as used herein, refer to a surgical stapler loaded with two or more staggered straight rows of staples, commonly used in abdominal surgery, thoracic surgery, gynecology, and pediatric surgery. Linear tissue staplers may be reusable or disposable, with distinctive designs for open surgeries and for endoscopic surgeries.

The terms ‘endoscopic cutter’, ‘endocutter’, ‘endo-cutter’, ‘endo cutter stapler’, and ‘cutting stapler’, as used herein, refer to a stapler with a blade provided between staples rows used for resection and transection of organs or tissues. The cutting stapler is loaded with two groups of staggered staples rows (each with a single row, or, double or triple staggered rows, or more, or any combination thereof), and simultaneously cuts and divides tissue between the two groups of rows in parallel to closing cut tissue ends with staples.

The term ‘fastener applying member’, as used herein, refers to a unit of the stapling jaw in a surgical stapler, designed to house a plurality of separate, individual staples (fasteners) or a cartridge of a plurality of staples (fasteners). A stapling jaw may include a single fastener applying member or a plurality of fastener applying members.

Hereinbelow are disclosed various aspects, and features thereof, of the present invention, as represented by illustrative descriptions of exemplary embodiments of a convertible linear tissue stapler, a method of surgically fastening or/and cutting tissue inside a body, and a method of deploying a linear tissue stapler to an operational form.

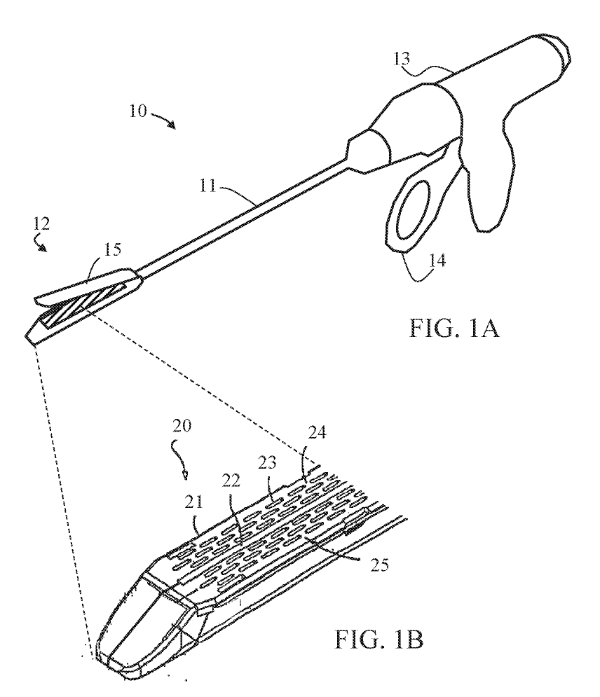

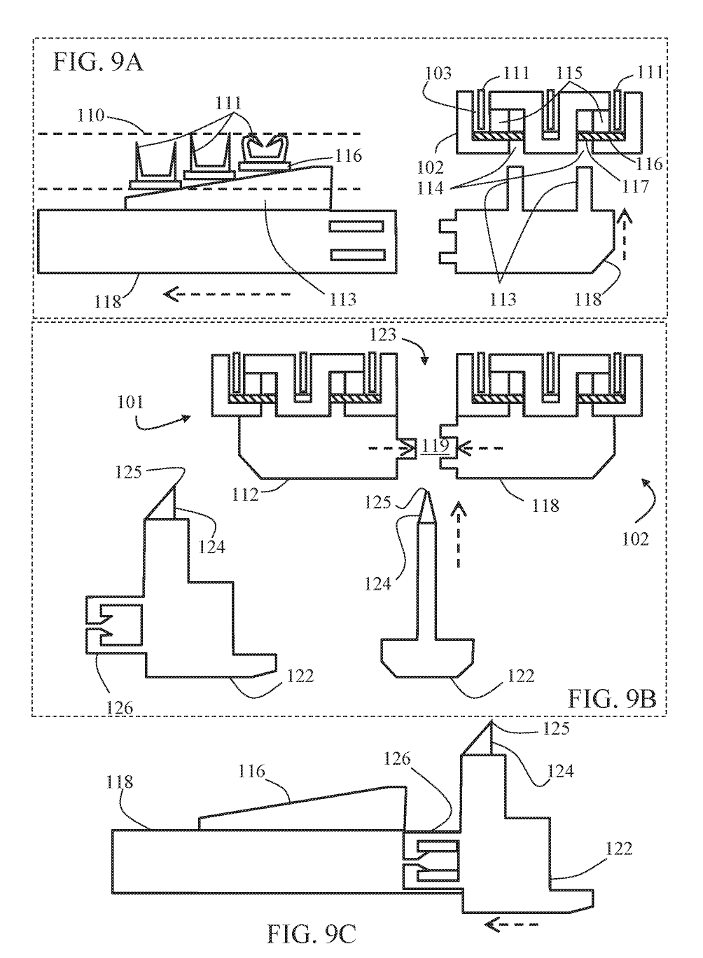

Reference is made to FIG. 1A, which schematically illustrates an isometric view of an exemplary embodiment of a cutting stapler 10, and to FIG. 1B, which schematically illustrates an enlarged partial view of an exemplary embodiment of a stapling jaw 20 of cutting stapler 10. Cutting stapler 10 includes an elongated body 11 connected at its distal end to a head 12 and at its proximal end to a handle assembly 13 that includes a stapling head actuating assembly 14. Head 12 includes a pair of opposing jaws, namely an anvil jaw 15 and stapling jaw 20, which are pivotally movable one with respect to the other and configured to grasp a tissue of allowed thickness therebetween. Stapling jaw 20 includes a first member, in a form of a first tissue fastener applying member 21, and a second member, in a form of a second tissue fastener applying member 25, shown in a deployment form, in which head 12 is operational for tissue stapling or/and cutting, with an elongated space 22 provided therebetween. Each tissue fastener applying member includes at least one row (e.g., three rows) 24 of staples which includes staggered grooved slots 23. Each grooved slot 23 houses a tissue staple and a staple pusher (e.g., staple 111 and staple pusher 116 as shown in FIG. 9A), the staple pusher capable of moving vertically in the corresponding grooved slot to press the corresponding tissue staple towards a corresponding depression in anvil jaw 15 (e.g., depressions 110 as shown in FIG. 8). A driver (e.g., driver 119 as shown in FIG. 9B) including a knife having a blade can be forced to pass lengthwise along elongated space 22 and by doing so generate at least one of the following end results: locally clasp adjacent portions of anvil jaw 15 and stapling jaw 20 about a tissue optionally provided therebetween, pushing staples pushers to eject staples toward anvil jaw 15 through the clasped tissue portion, and cut the tissue along elongated space 22 between the jaws, with the blade. Upon actuation of stapling head actuating assembly 14, the jaws approximate and the driver is pushed distally. Optionally and alternatively, tissue grasping and driver actuation are initiated using separate actuators.

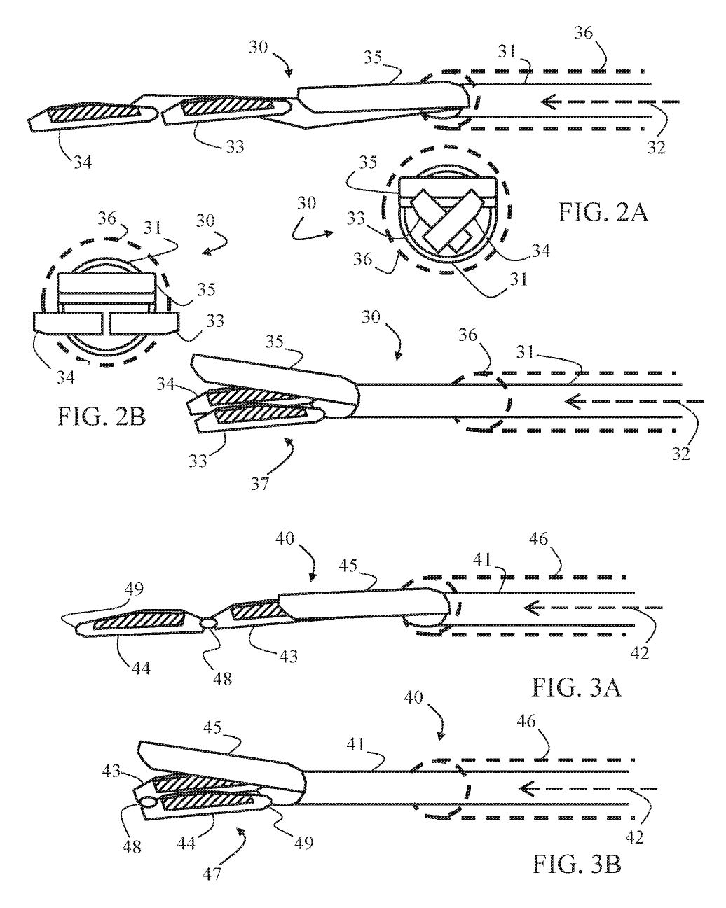

Referring is now made to FIGS. 2A-2B which schematically illustrate exemplary embodiments of a delivery configuration and a deployment configuration, respectively, of a linear tissue stapler 30, which may be in a form of an endo cutter stapler. Linear tissue stapler 30 includes an elongated body 31 having a longitudinal axis 32, and a first member (first tissue fastener applying member) 33 and a second member (second tissue fastener applying member) 34 positioned distally to elongated body 31. In some embodiments, at least one of first tissue fastener applying member 33 and second tissue fastener applying member 34 includes at least one row of tissue fasteners (e.g., similar to row 24 of staples as shown in FIG. 1B). An anvil jaw 35 is pivotally connected to elongated body 31 and is operable with jaws actuating means (e.g., stapling head actuating assembly 14 as shown in FIG. 1A) provided proximally to elongated body 31.

In some embodiments, linear tissue stapler 30 is convertible from a delivery configuration (illustrates in FIG. 2A), in which linear tissue stapler 30 is passable through a passage 36 enclosing a minimal inner diameter, to a deployment configuration (illustrated in FIG. 2B), in which linear tissue stapler 30 is operational. Passage 36 may be, for example, a lumen enclosed by a laparoscopic sheath, port or trocar, or, optionally and alternatively, a lumen enclosed by an endoscopic channel. The minimal inner diameter may be equal to or less than about 20 mm, optionally, equal to or less than about 8 mm, optionally, equal to or less than about 6 mm, optionally, equal to or less than about 4 mm, or larger, or smaller, or an intermediate size.

In some embodiments, in the delivery configuration, first tissue fastener applying member 33 and second tissue fastener applying member 34 are consecutively arranged lengthwise, relative to longitudinal axis 32, such that an overall diameter thereof is smaller than the minimal inner diameter (enclosed by passage 36). Optionally and additionally, in the deployment configuration, first tissue fastener applying member 33 and second tissue fastener applying member 34 are juxtapositionally arranged relative to longitudinal axis 32 such that an overall diameter thereof is equal to or greater than the minimal inner diameter (enclosed by passage 36).

In some embodiments, in the deployment configuration, first tissue fastener applying member 33 and second tissue fastener applying member 34 are interlocked with each other to rigidly form a single stapling jaw 37. In some embodiments, in the deployment configuration, anvil jaw 35 opposes stapling jaw 37, wherein stapling jaw 37 and anvil jaw 35 are movable with respect to each other from an open position, in which the jaws are spaced apart, to a closed position, in which the jaws are in close proximity to each other.

FIGS. 3A-3B schematically illustrate exemplary embodiments of a delivery configuration and a deployment configuration, respectively, of a linear tissue stapler 40. Linear tissue stapler 40 includes an elongated body 41 having a longitudinal axis 42, and a first member (first tissue fastener applying member) 43 and a second member (second tissue fastener applying member) 44 positioned distally to elongated body 41. In some embodiments, at least one of first tissue fastener applying member 43 and second tissue fastener applying member 44 includes at least one row of tissue fasteners (e.g., similar to rows 24 of staples as shown in FIG. 1B). An anvil jaw 45 is pivotally connected to elongated body 41 and is operable with jaws actuating means (e.g., stapling head actuating assembly 14 as shown in FIG. 1A) provided proximally to elongated body 41.

In some embodiments, linear tissue stapler 40 is convertible from a delivery configuration (illustrated in FIG. 3A), in which linear tissue stapler 40 is passable through a passage 46 enclosing a minimal inner diameter, to a deployment configuration (illustrated in FIG. 3B), in which linear tissue stapler 40 is operational. Passage 46 may be, for example, a lumen enclosed by a laparoscopic sheath, port or trocar, or, optionally and alternatively, a lumen enclosed by an endoscopic channel. The minimal inner diameter may be equal to or less than about 20 mm, optionally, equal to or less than about 12 mm, optionally, equal to or less than about 8 mm, optionally, equal to or less than about 6 mm, optionally, equal to or less than about 4 mm, or larger, or smaller, or an intermediate size.

In some embodiments, in the delivery configuration, first tissue fastener applying member 43 and second tissue fastener applying member 44 are consecutively arranged lengthwise, relative to longitudinal axis 42, such that an overall diameter thereof is smaller than the minimal inner diameter (enclosed by passage 46). Optionally and additionally, in the deployment configuration, first tissue fastener applying member 43 and second tissue fastener applying member 44 are juxtapositionally arranged relative to longitudinal axis 42 such that an overall diameter thereof is equal to or greater than the minimal inner diameter (enclosed by passage 46).

In some embodiments, first tissue fastener applying member 43 is hingedly connected to second tissue fastener applying member 44, optionally with a hinge 48, such that second tissue fastener applying member 44 is rotatable with respect to first tissue fastener applying member 43. In some embodiments, in the delivery configuration, a second end 49 of second tissue fastener applying member 44 is positioned distally to the distal end of first tissue fastener applying member 43, and in the deployment configuration, second end 49 of second tissue fastener applying member 44 is positioned proximally to distal end of first tissue fastener applying member 43. In some embodiments, first tissue fastener applying member 43 shifts transversally upon juxtaposing to second tissue fastener applying member 44.

In some embodiments, in the deployment configuration, first tissue fastener applying member 43 and second tissue fastener applying member 44 are interlocked with each other to rigidly form a single stapling jaw 47. In some embodiments, in the deployment configuration, anvil jaw 45 opposes stapling jaw 47, wherein stapling jaw 47 and anvil jaw 45 are movable with respect to each other from an open position, in which the jaws are spaced apart, to a closed position, in which the jaws are in close proximity to each other.

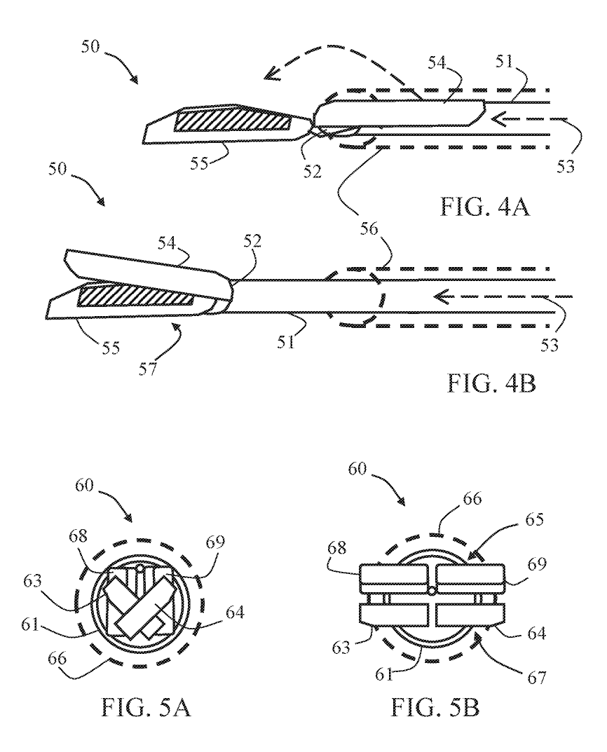

FIGS. 4A-4B schematically illustrate exemplary embodiments of a delivery configuration and a deployment configuration, respectively, of a linear tissue stapler 50. Linear tissue stapler 50 includes an elongated body 51, having a distal end 52 and a longitudinal axis 53, a first member (anvil jaw) 54, and a second member (first tissue fastener applying member) 55 including at least one row of tissue fasteners (e.g., similar to rows 24 of staples as shown in FIG. 1B). Anvil jaw 54 is pivotally connected to elongated body 51 and is operable with jaws actuating means (e.g., stapling head actuating assembly 14 as shown in FIG. 1A) provided proximally to elongated body 51.

In some embodiments, linear tissue stapler 50 is convertible from a delivery configuration, in which linear tissue stapler 50 is passable through a passage 56 enclosing a minimal inner diameter, to a deployment configuration, in which linear tissue stapler 50 is operational. Passage 56 may be, for example, a lumen enclosed by a laparoscopic sheath, port or trocar, or, optionally and alternatively, a lumen enclosed by an endoscopic channel. The minimal inner diameter may be equal to or less than about 20 mm, optionally, equal to or less than about 12 mm, optionally, equal to or less than about 8 mm, optionally, equal to or less than about 6 mm, optionally, equal to or less than about 4 mm, or larger, or smaller, or an intermediate size.

In some embodiments, anvil jaw 54 is provided proximally to distal end 52 of elongated body 51, in the delivery configuration, and is shiftable to a deployed position, in the deployment configuration, being distally to distal end 52 of elongated body 51. In some embodiments, in the deployment configuration, first tissue fastener applying member 55 interlocks with distal end 52 of elongated body 51 to rigidly form a single stapling jaw 57. In some embodiments, in the deployment configuration, anvil jaw 54 opposes stapling jaw 57, wherein stapling jaw 57 and anvil jaw 54 are movable with respect to each other from an open position, in which the jaws are spaced apart, to a closed position, in which the jaws are in close proximity to each other.

In some embodiments, in the delivery configuration, anvil jaw 54 and first tissue fastener applying member 55 are consecutively arranged lengthwise relative to longitudinal axis 53 such that an overall diameter thereof is smaller than the minimal inner diameter (enclosed by passage 56). In some embodiments, in the deployment configuration, anvil jaw 54 and first tissue fastener applying member 55 are juxtapositionally arranged relative to longitudinal axis 53 such that an overall diameter thereof is equal to or greater than the minimal inner diameter (enclosed by passage 56).

FIGS. 5A-5B schematically illustrate front view of exemplary embodiments of a delivery configuration and a deployment configuration, respectively, of a linear tissue stapler 60. Linear tissue stapler 60 includes an elongated body 61 having a longitudinal axis, and a first member (first tissue fastener applying member) 63 and a second member (second tissue fastener applying member) 64 positioned distally to elongated body 61. In some embodiments, at least one of first tissue fastener applying member 63 and second tissue fastener applying member 64 includes at least one row of tissue fasteners (e.g., similar to rows 24 of staples as shown in FIG. 1B). An anvil jaw 65 is pivotally connected to elongated body 61 and is operable with jaws actuating means (e.g., stapling head actuating assembly 14 as shown in FIG. 1A) provided proximally to elongated body 61.

In some embodiments, linear tissue stapler 60 is convertible from a delivery configuration (illustrates in FIG. 5A), in which linear tissue stapler 60 is passable through a passage 66 enclosing a minimal inner diameter, to a deployment configuration (illustrated in FIG. 5B), in which linear tissue stapler 60 is operational. Passage 66 may be, for example, a lumen enclosed by a laparoscopic sheath, port or trocar, or, optionally and alternatively, a lumen enclosed by an endoscopic channel. The minimal inner diameter may be equal to or less than about 20 mm, optionally, equal to or less than about 12 mm, optionally, equal to or less than about 8 mm, optionally, equal to or less than about 6 mm, optionally, equal to or less than about 4 mm, or larger, or smaller, or an intermediate size.

In some embodiments, in the delivery configuration, first tissue fastener applying member 63 and second tissue fastener applying member 64 are consecutively arranged lengthwise, relative to the longitudinal axis of elongated body 61, such that an overall diameter thereof is smaller than the minimal inner diameter (enclosed by passage 66). Optionally and additionally, in the deployment configuration, first tissue fastener applying member 63 and second tissue fastener applying member 64 are juxtapositionally arranged relative to the longitudinal axis of elongated body 61 such that an overall diameter thereof is equal to or greater than the minimal inner diameter (enclosed by passage 66).

In some embodiments, anvil jaw 65 includes convertible anvil members, such as a first anvil member 68 pivotally connected with a second anvil member 69. In some embodiments, the anvil members are configured to shift from a first position in the delivery configuration, such that an overall width thereof is equal to or smaller than the minimal inner diameter (enclosed by passage 66), to a second position in the deployment configuration, such that an overall width thereof is greater than the minimal inner diameter (enclosed by passage 66).

In some embodiments, in the deployment configuration, first tissue fastener applying member 63 and second tissue fastener applying member 64 are interlocked with each other to rigidly form a single stapling jaw 67. In some embodiments, in the deployment configuration, anvil jaw 65 opposes stapling jaw 67, wherein stapling jaw 67 and anvil jaw 65 are movable with respect to each other from an open position, in which the jaws are spaced apart, to a closed position, in which the jaws are in close proximity to each other.

In some embodiments, a linear tissue stapler according to the present disclosure (for example any of staplers 10, 30, 40, 50 and 60) includes a first track, wherein in the delivery configuration the first tissue fastener applying member contacts the first track at a first location, and in the deployment configuration, the first tissue fastener applying member contacts the first track at a second location. Optionally and additionally, the linear tissue stapler includes a second track, wherein in the delivery configuration the second tissue fastener applying member contacts the second track at a third location, and in the deployment configuration, the second tissue fastener applying member contacts the second track at a fourth location. In some embodiments, the first track or/and the second track is located within a lumen of the elongated body. In some embodiments, the first track is configured such that the first tissue fastener applying member can travel lengthwise or/and rotate relative to the longitudinal axis upon converting between the delivery configuration and the deployment configuration. Optionally and additionally, the second track is configured such that the second tissue fastener applying member can travel lengthwise or/and rotate relative to the longitudinal axis upon converting between the delivery configuration and the deployment configuration.

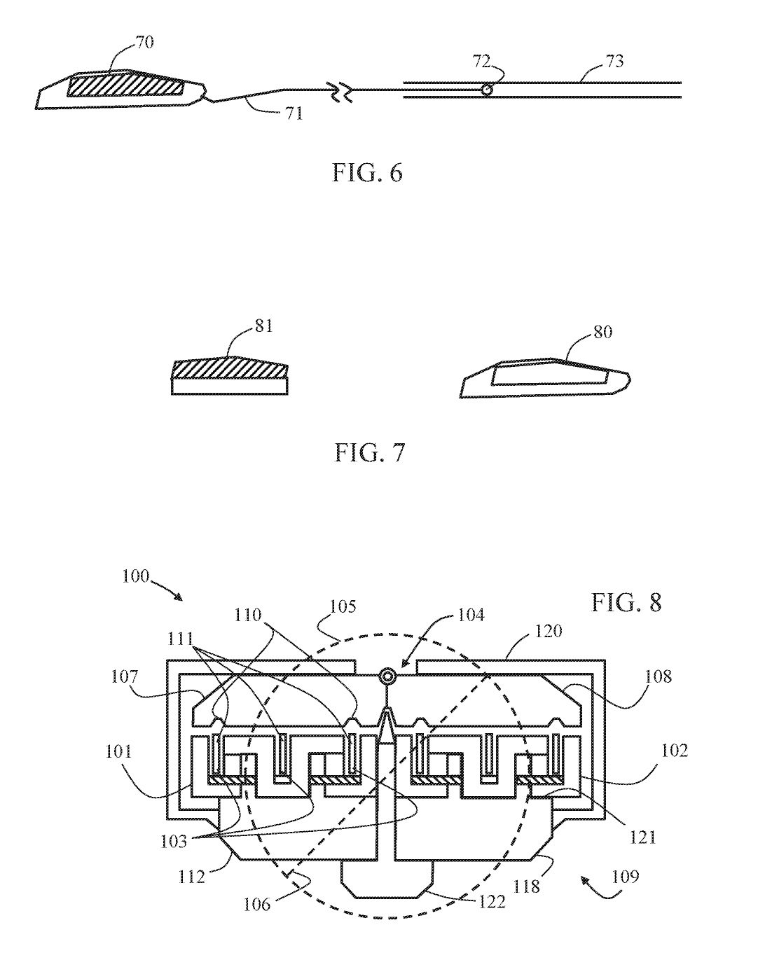

Referring back to the figures, FIG. 6 shows a tissue fastener applying member 70 connected with a rod 71 to a track slider 72. Slider 72 is movable in a track 73 which is substantially straight and configured such that tissue fastener applying member 70 can travel lengthwise relative to a longitudinal axis of a stapler elongated body (e.g., as shown in FIGS. 4A-4B), upon converting between the delivery configuration and the deployment configuration. Optionally and alternatively, the track is substantially helical and configured such that tissue fastener applying member can travel lengthwise and rotate relative to a longitudinal axis of a stapler elongated body, upon converting between the delivery configuration and the deployment configuration.

In some embodiments, a linear tissue stapler according to the present disclosure (for example any of staplers 10, 30, 40, 50, 60, 70 and 75) includes tissue fastener applying members, wherein each of the tissue fastener applying members includes a linear staples cartridge including a plurality of tissue staples. Optionally, the linear staples cartridges are replaceable or/and ejectable from the tissue fastener applying members. FIG. 7 schematically illustrates an exemplary embodiment of a tissue fastener applying member 80 configured for deployment with a replaceable staples cartridge 81.

Reference is now made to FIG. 8, which schematically illustrates a transverse cut view of an exemplary embodiment of a stapling head 100 of an exemplary convertible linear tissue stapler in a form of an endo cutter stapler, and to FIGS. 9A-9C which schematically illustrate exemplary embodiments of components of stapling head 100. Stapling head 100 is connected or connectable to an elongated body (e.g., elongated body 11 as shown in FIG. 1A) having a longitudinal axis, and includes a first member (first tissue fastener applying member) 101 and a second member (second tissue fastener applying member) 102 positioned distally to the elongated body. Each of first tissue fastener applying member 101 and second tissue fastener applying member 102 includes three staggered rows 103 of tissue fasteners 111. Each row 103 includes at least 5 fasteners, optionally at least 10 fasteners, optionally at least 15 fasteners, or higher, or lower, or an intermediate number. In some embodiments, each of the tissue fastener applying members includes or can be filled with a linear staples cartridge including a plurality of tissue staples (i.e., the fasteners are in a form of tissue staples). Optionally, the linear staples cartridges are replaceable or/and ejectable from the tissue fastener applying members. Stapling head 100 may be coupled with different types or/and sizes of staples cartridges including but not limited to cartridges containing rows of 8×30 mm staples, rows of 11×45 mm staples, rows of 10×45 mm staples, or rows of 14×60 mm staples.

In some embodiments, the tissue fasteners (e.g., staples) have staples legs plastically deformable to an inwardly bent form following compressing thereof between the stapling jaw and the anvil jaw. Optionally, at least one staple has leg size being greater than 3 mm in length, optionally about 3.5 mm, or optionally about 3.8 mm, or optionally about 4.1 mm, in length. Optionally, alternatively or additionally, at least one staple has a maximal leg size smaller than 3 mm in length, optionally about 2.5 mm or optionally about 2 mm, in length.

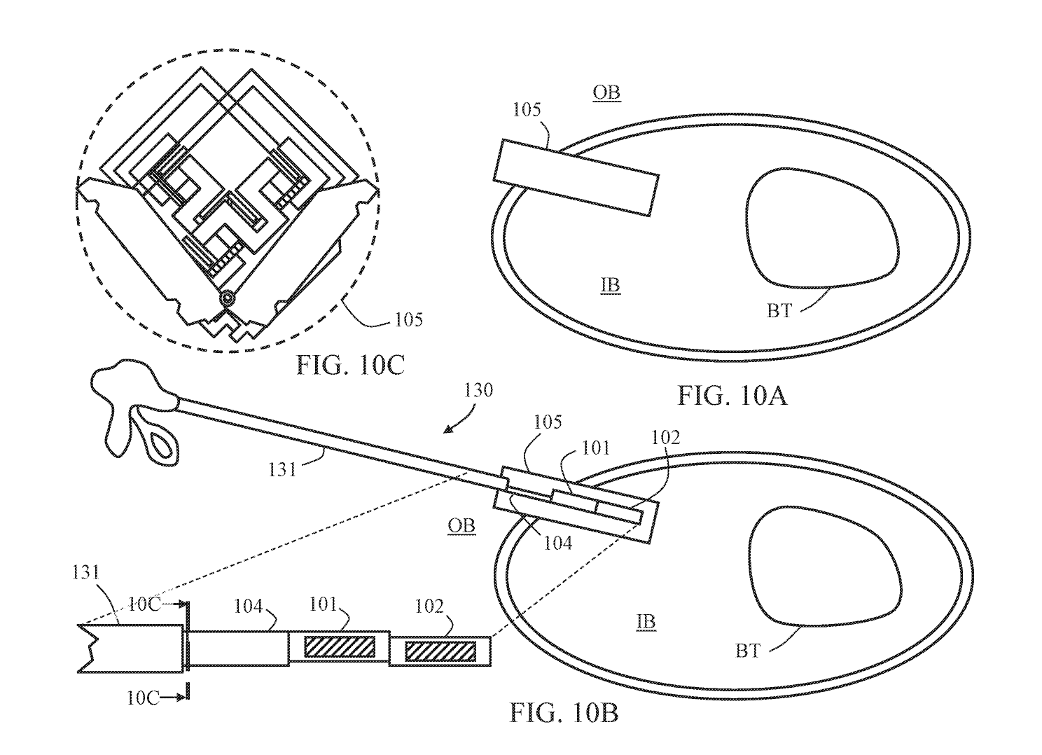

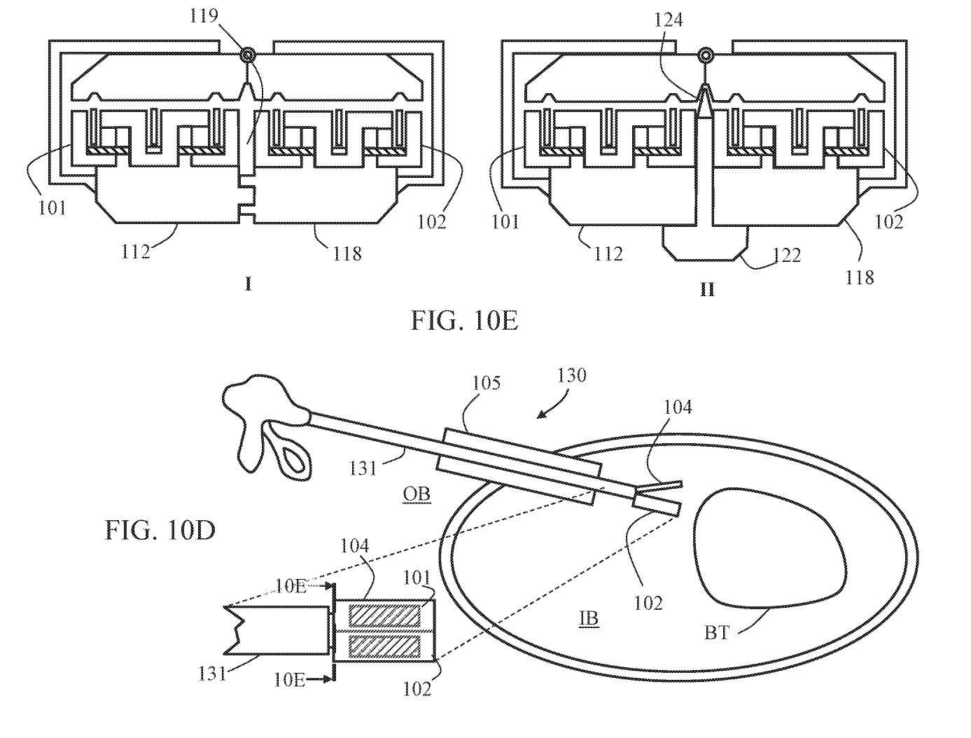

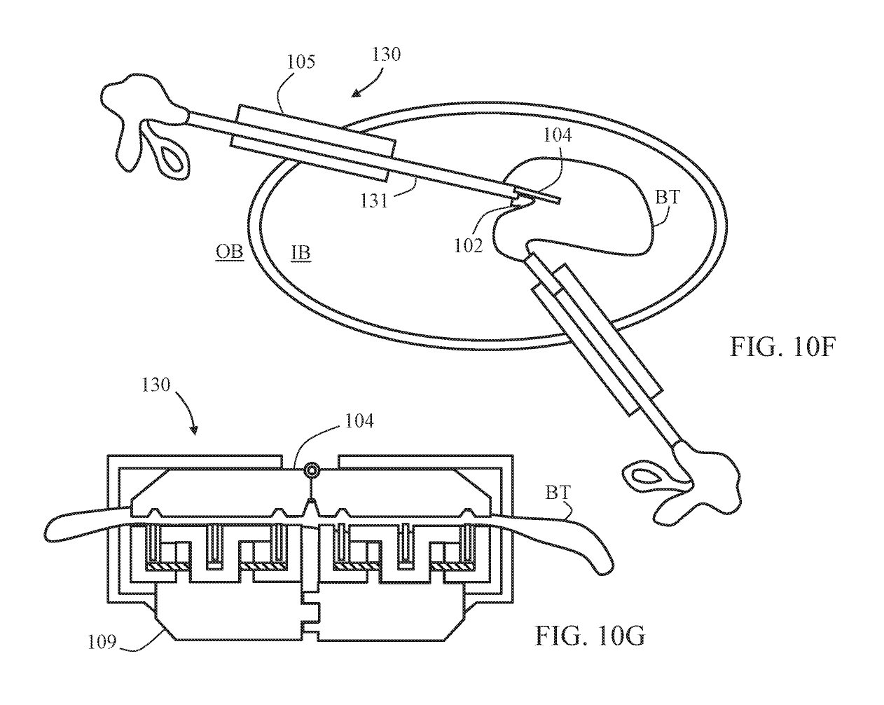

In some embodiments, stapling head 100, as part of the linear tissue stapler, is convertible from a delivery configuration (e.g., as shown in FIG. 10C), in which the linear tissue stapler is passable through a passage 105 enclosing a minimal inner diameter 106, to a deployment configuration, in which the linear tissue stapler is operational. Passage 105 may be, for example, enclosed by a laparoscopic sheath, port or trocar, an endoscopic channel or, optionally and alternatively, a lumen enclosed by a natural bodily orifice. Minimal inner diameter 106 may be equal to or less than about 20 mm, optionally, equal to or less than about 12 mm, optionally, equal to or less than about 8 mm, optionally, equal to or less than about 6 mm, optionally, equal to or less than about 4 mm, or larger, or smaller, or an intermediate size. FIG. 8 illustrates stapling head 100 at the deployment configuration, in which first tissue fastener applying member 101 and second tissue fastener applying member 102 are juxtapositionally arranged (relative to the longitudinal axis of the elongated body) such that an overall diameter thereof is equal to or greater than minimal inner diameter 106.

An anvil jaw 104 is pivotally connected to the elongated body and is operable with a stapling head actuating assembly (e.g., similar to stapling head actuating assembly 14 as shown in FIG. 1A) provided proximally to the elongated body. In some embodiments, anvil jaw 104 includes convertible anvil members, including a first anvil member 107 pivotally connected with a second anvil member 108. In some embodiments, the anvil members are configured to shift from a first position in the delivery configuration (e.g., as shown in FIG. 10C), such that an overall width thereof is equal to or smaller than minimal inner diameter 106, to a second position in the deployment configuration (as shown in FIG. 8), such that an overall width thereof is greater than minimal inner diameter 106.

FIG. 8 illustrates stapling head 100 in the deployment configuration, in which first tissue fastener applying member 101 and second tissue fastener applying member 102 are interlocked with each other to rigidly form a single stapling jaw 109. In some embodiments, in the deployment configuration, anvil jaw 104 opposes stapling jaw 109, wherein stapling jaw 109 and anvil jaw 104 are movable with respect to each other from an open position, in which the jaws are spaced apart, to a closed position, in which the jaws are in close proximity to each other. Anvil jaw 104 includes a plurality of depressions 110 positionable opposably to a corresponding number of fasteners 111 such that upon clamping anvil jaw 104 and fasteners jaw 109 together a fastener 111 is forced to bend to a closed shape against an opposing depression 110.

As also shown in more details in FIGS. 9A and 9B, each of the tissue fastener applying members 101 and 102 includes a driving member, in this particular example a first driving member 112 and a second driving member 118, respectively, each including a number of runners 113. Each runner 113 is slidable along a corresponding grooved route 114 provided along a length of the corresponding tissue fastener applying member 101 or 102. In some embodiments, each row 103 of tissue fasteners/staples 111 includes a number of grooved slots 115. Each grooved slot 115 houses a staple pusher 116 with a cam head 117, such that upon sliding of the sliding runners 113 distally through grooved routs 114, each staple pusher 116 moves vertically in the corresponding grooved slot 115 to press the corresponding tissue staple 111 towards a corresponding depression 110 in anvil jaw 104.

In some embodiments, in the deployment configuration, first driving member 112 of first tissue fastener applying member 101 and second driving member 118 of second tissue fastener applying member 102 are interlocked with each other to form a single driver 119 (shown in more detail, in FIG. 9B). In some embodiments, at least one driving member 112 or/and 118 includes or is connected to a top plate 120, pressing against a surface area of a corresponding tissue fastener applying member 101 or/and 102. Optionally, top plate 120 is interconnected with a bottom plate 121 pressing against an opposing surface area of anvil jaw 104. Top plate 120 and bottom plate 121 are optionally elastically connected or fixedly distanced with respect to each other such that, at any location of driving member 119 along the tissue fastener applying member length, the opposing surface areas of the corresponding tissue fastener applying member 101 or/and 102 and anvil jaw 104 are clamped by top plate 120 and bottom plate 121. Optionally, a substantially fixed distance is maintained between anvil jaw 104 and fasteners jaw 109, at least between opposing surfaces thereof covered by top plate 120 and bottom plate 121, the fixed distance optionally falls within a range of between about 0.5 mm and about 3 mm, optionally, between about 1 mm and about 2 mm, or larger, or smaller, or an intermediate distance. Optionally, upon clamping of the opposing surface areas of the corresponding tissue fastener applying members 101 and 102, and anvil jaw 104, at least one tissue staple 111 provided between the opposing areas is pressed towards a corresponding depression 110 in anvil jaw 104.

In some embodiments (e.g., as shown in FIGS. 8, 9B, and 9C), stapling head 100, as part of the linear tissue stapler, includes a driving clutch member 122 engageable with driving members 112 and 118 (in this example, using snap lock 126), wherein, in the delivery configuration, driving clutch member 122 is disengaged from the driving members (as shown in FIG. 9B), and in the deployment configuration (as shown in FIGS. 8 and 9C), driving clutch member 122 is engaged with the driving members 112 and 118. Driving clutch member 122 is optionally operable with clutch actuating means which may be provided proximally to the elongated body, which are configured to drive driving clutch member 122 lengthwise along the longitudinal axis in a spaced passage 123 between first tissue fastener applying member 101 and second tissue fastener applying member 102, when in the deployment configuration.

In some embodiments, particularly when the linear tissue stapler is configured as an endo-cutter stapler, driving clutch member 122 includes or is connected or connectable to a blade 124, with a sharp edge 125, extending between jaws 104 and 109 when in the deployment configuration. As such, sharp edge 125 is configured to cut through a tissue clasped between the jaws upon distal sliding of driving clutch member 122 along spaced passage 123.

In some embodiments, driving clutch member 122 is positioned between the elongated body and first tissue fastener applying member 101, in the delivery configuration. Optionally and alternatively driving clutch member 122 is positioned in a lumen of the elongated body and proximally to first tissue fastener applying member 101, in the delivery configuration.

In order to utilize a convertible linear stapler, such as the one partly shown in FIGS. 8 and 9A-9C, the stapler is provided such that first tissue fastener applying member 101 and second tissue fastener applying member 102, which are positioned distally to the elongated body of the stapler (e.g., similar to that shown in FIG. 2A), are consecutively arranged lengthwise relative to a longitudinal axis of the elongated body. The repositioning optionally increases a diameter of a circumscribed circle around a maximal transverse cross section of the linear stapler, which may increase from about 6 mm or less to over 6 mm, or from about 10 mm or less to over 10 mm, or larger, or smaller, or an intermediate size, such as, for example, from about 5.5 mm to about 10 or to about 15 mm.

First tissue fastener applying member 101 or/and second tissue fastener applying member 102 are then repositioned to be juxtapositionally arranged relative to the longitudinal axis. In some embodiments, the repositioning includes distally shifting first tissue fastener applying member 101 or/and second tissue fastener applying member 102 to proximity of the elongated body, or/and rotating any or both of them about a corresponding longitudinal axis thereof.

Then, first tissue fastener applying member 101 and second tissue fastener applying member 102 are interlocked with each other to rigidly form stapling jaw 119, movable with respect to anvil jaw 104, from an open position, in which the jaws are spaced apart, to a closed position, in which the jaws are in close proximity to each other. In some embodiments, the interlocking includes connecting first driving member 112 and second driving member 118 to form a single driver.

Driving clutch member 122 may be provided proximally to the elongated body and be operable with clutch actuating means. Driving clutch member 122 may then be engaged with the both driving members 112 and 118 and driven (by the clutch actuating means) lengthwise along the longitudinal axis in spaced passage 123.

Reference is made to FIGS. 10A-10H, which schematically illustrate exemplary embodiments of different scenarios representing possible exemplary steps in a method of deploying a convertible linear tissue stapler 130, including stapling head 100, and surgically affecting a tissue BT in a body. As shown in FIG. 10A, passage 105 (optionally enclosed with a laparoscopic port) is provided for connecting between an outer body environment OB and an in-body location IB in vicinity of tissue BT.

As shown in FIG. 10B, linear tissue stapler 130 is passed through passage 105 towards in-body location IB. Linear tissue stapler 130 includes an elongated body 131. First tissue fastener applying member 101 and second tissue fastener applying member 102 are positioned distally to elongated body 131 and arranged in a delivery configuration in which first tissue fastener applying member 101 and second tissue fastener applying member 102 are consecutively arranged lengthwise relative to a longitudinal axis 132 of elongated body 131. Optionally, anvil jaw 104 is provided between the tissue fastener applying members 101 and 102 and elongated body 131 in the delivery configuration, as shown in FIG. 10B. FIG. 10C illustrates a rear view of stapling head 100, demonstrating how its components are arranged such to pass through passage 105.

First tissue fastener applying member 101 and second tissue fastener applying member 102 can then be emerged out of passage 105 within the body, allowing for linear tissue stapler 130 to be converted (inside the body) from the delivery configuration to the deployment configuration (as shown in FIGS. 10D and 8), in which first tissue fastener applying member 101 and second tissue fastener applying member 102 are juxtapositionally arranged relative to longitudinal axis 132 and interlocked with each other to rigidly form stapling jaw 109.

In some embodiments, converting linear tissue stapler 130 to the deployment configuration includes or is followed by providing blade 124 in spaced passage 119 between first tissue fastener applying member 101 and second tissue fastener applying member 102 (as shown in FIGS. 10E and 9B). In some embodiments, the converting also includes connecting first driving member 112 and second driving member 118 to form a single driver.

FIGS. 10F and 10G show tissue BT being clasped between stapling jaw 109 and anvil jaw 104, and, as shown in FIGS. 10H and 101, linear tissue stapler 130 is employed such that stapling jaw 109 releases a plurality of fasteners 111 through tissue BT towards anvil jaw 104. This employing includes or is followed by sliding blade 124 along spaced passage 119 to cut through the clasped tissue BT with sharp edge 125.

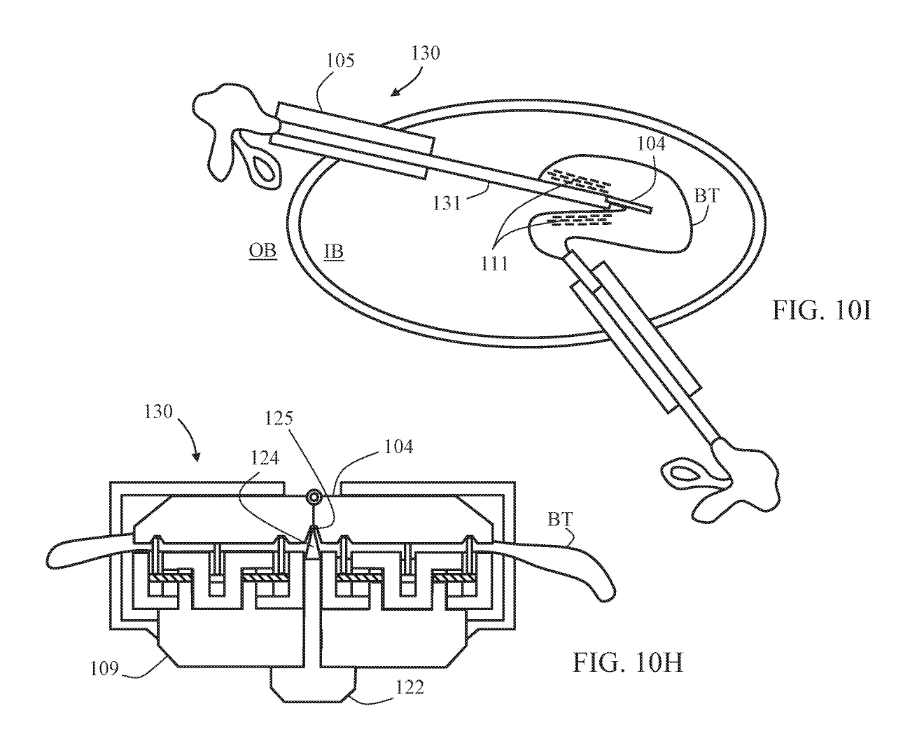

FIGS. 11A-11B schematically illustrate exemplary embodiments of a delivery configuration and a deployment configuration, respectively, in side views of an exemplary linear tissue stapler 230. Linear tissue stapler 230 includes an elongated body 231 extending along a longitudinal axis 233 and having a distal end 232, a first member (anvil jaw) 234, and a second member (stapling jaw) 235 containing parallel rows of tissue fasteners (e.g., similar to rows 24 of staples as shown in FIG. 1B). When in a fully operational mode, the anvil jaw 234 is pivotally connected to elongated body 231 or to stapling jaw 235, and is operable with jaws actuating means (e.g., stapling head actuating assembly 14 as shown in FIG. 1A) provided proximally to elongated body 231.

Linear tissue stapler 230 is convertible from a delivery configuration (FIG. 11A) to a deployment configuration (FIG. 11B). When in the delivery configuration, linear tissue stapler 230 is sized to pass through a passage 236 enclosing a minimal inner diameter 238. Passage 236 may be, for example, a lumen enclosed by a laparoscopic sheath, port or trocar, or, optionally and alternatively, a lumen enclosed by an endoscopic channel. The minimal inner diameter may be equal to or less than about 20 mm, optionally, equal to or less than about 12 mm, optionally, equal to or less than about 8 mm, optionally, equal to or less than about 6 mm, optionally, equal to or less than about 4 mm, or larger, or smaller, or an intermediate size. As shown, anvil jaw 234 and stapling jaw 235 are consecutively arranged lengthwise whereby a maximal stapler cross-sectional dimension 239 is smaller than minimal inner diameter 238. When in the delivery configuration, anvil jaw is positioned entirely proximally to elongated body distal end 232. As such, stapler 230 is absent of an operational stapler head (i.e., it does not have a functional head capable of performing at least one of grasping, cutting and stapling to a body tissue).

When in the deployment configuration, anvil jaw 234 is positioned mostly or entirely distally to elongated body distal end 232 and is juxtapositionally arranged with stapling jaw 235 forming an operational stapler head 237. In the deployment configuration the anvil jaw 234 overlaps (i.e., coincides partially or wholly) with stapling jaw 235. Stapling jaw 235 is optionally coupled to elongated body distal end 232 and lying distally thereto in both delivery configuration and deployment configuration. Anvil jaw optionally interlocks with elongated body distal end 232 and/or with stapling jaw 235 in the deployment configuration to form operational stapler head 237 distally adjacent elongated body distal end 232. When in the form of operational stapler head 237, the stapling jaw 235 and anvil jaw 234 are movable with respect to each other from an open position, wherein the jaws are spaced apart, to a closed position (FIG. 11B), wherein the jaws are in close proximity to each other.

The maximal stapler cross-sectional dimension 239′ in the deployment configuration is equal to or greater than minimal inner diameter 238. Cross-sectional dimensions 239 and 239′ may be a maximal height, a maximal width and/or a maximal diameter. The maximal stapler cross-sectional dimension in the deployment configuration is equal to or greater than about 3 mm, optionally, equal to or greater than about 5 mm, optionally, equal to or greater than about 8 mm, optionally, equal to or greater than about 12 mm, optionally, equal to or greater than about 20 mm, optionally, equal to or greater than about 30 mm, or larger, or smaller, or an intermediate value.

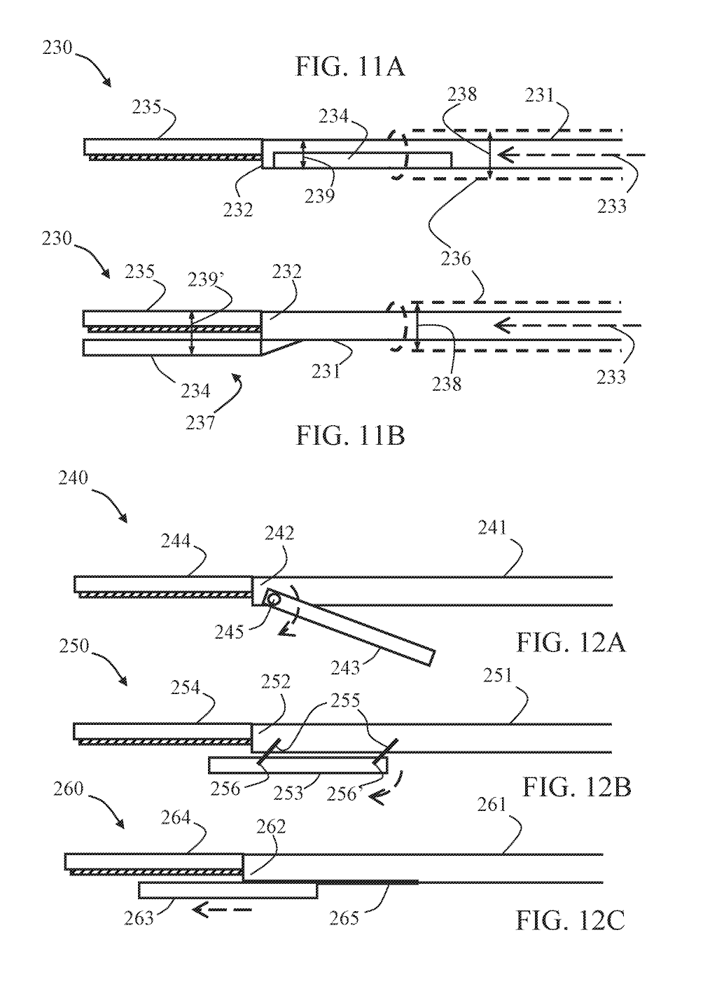

Reference is made to FIGS. 12A-12C, which schematically illustrate side views of exemplary embodiments of different shifting mechanisms between a delivery configuration and a deployment configuration of exemplary linear tissue staplers 240, 250 and 260. Linear tissue stapler 240 (FIG. 12A) includes an elongated body 241 with an elongated body distal end 242, an anvil jaw 243, and a stapling jaw 244 containing parallel rows of tissue fasteners (e.g., similar to rows 24 of staples as shown in FIG. 1B). Linear tissue stapler 240 is convertible from a delivery configuration (similar to that shown in FIG. 11A) to a deployment configuration (e.g., similar to that shown in FIG. 11B). When in the delivery configuration, linear tissue stapler 240 is sized to pass through a passage enclosing a minimal inner diameter, such a lumen enclosed by a laparoscopic sheath, port or trocar, or an endoscopic channel. The minimal inner diameter may be equal to or less than about 20 mm, optionally, equal to or less than about 12 mm, optionally, equal to or less than about 8 mm, optionally, equal to or less than about 6 mm, optionally, equal to or less than about 4 mm, or larger, or smaller, or an intermediate size. Anvil jaw 243 and stapling jaw 244 may be consecutively arranged lengthwise whereby their maximal cross-sectional dimension is smaller than minimal inner diameter. When in the delivery configuration, anvil jaw 243 may be positioned entirely proximally to elongated body distal end 242 making stapler 240 absent of an operational stapler head. When in the deployment configuration, anvil jaw 243 is positioned mostly or entirely distally to elongated body distal end 242 and is juxtapositionally arranged with stapling jaw 244 to form an operational stapler head. In the deployment configuration the anvil jaw 243 may oppose and/or overlap with stapling jaw 244.

Stapling jaw 244 is optionally coupled to elongated body distal end 242 and lying distally thereto in both delivery configuration and deployment configuration. Anvil jaw 243 is configured to swivel towards stapling jaw 244 when shifting between the delivery configuration and the deployment configuration, as shown in FIG. 12A. Swiveling may be actuated about a pivot point provided at or adjacent elongated body distal end 242, optionally facilitated by a hinge 245, as shown. Anvil jaw 243 may then interlock with elongated body distal end 242 and/or with stapling jaw 244 in the deployment configuration to form the operational stapler head distally adjacent elongated body distal end 242. Stapling jaw 244 and anvil jaw 243 are then movable with respect to each other from an open position, wherein the jaws are spaced apart, to a closed position, wherein the jaws are in close proximity to each other. The maximal stapler cross-sectional dimension in the deployment configuration is equal to or greater than minimal inner diameter, and may be a maximal height, a maximal width and/or a maximal diameter. The maximal stapler cross-sectional dimension in the deployment configuration is equal to or greater than about 3 mm, optionally, equal to or greater than about 5 mm, optionally, equal to or greater than about 8 mm, optionally, equal to or greater than about 12 mm, optionally, equal to or greater than about 20 mm, optionally, equal to or greater than about 30 mm, or larger, or smaller, or an intermediate value.

Linear tissue stapler 250 (FIG. 12B) includes an elongated body 251 with an elongated body distal end 252, an anvil jaw 253, and a stapling jaw 254 containing parallel rows of tissue fasteners. Linear tissue stapler 250 is convertible from a delivery configuration (e.g., similar to that shown in FIG. 11A) to a deployment configuration (e.g., similar to that shown in FIG. 11B). When in the delivery configuration, linear tissue stapler 250 is sized to pass through a passage enclosing a minimal inner diameter, such a lumen enclosed by a laparoscopic sheath, port or trocar, or an endoscopic channel. The minimal inner diameter may be equal to or less than about 20 mm, optionally, equal to or less than about 12 mm, optionally, equal to or less than about 8 mm, optionally, equal to or less than about 6 mm, optionally, equal to or less than about 4 mm, or larger, or smaller, or an intermediate size. Anvil jaw 253 and stapling jaw 254 may be consecutively arranged lengthwise whereby their maximal cross-sectional dimension is smaller than minimal inner diameter. When in the delivery configuration, anvil jaw 253 may be positioned entirely proximally to elongated body distal end 252 making stapler 250 absent of an operational stapler head. When in the deployment configuration, anvil jaw 253 is positioned mostly or entirely distally to elongated body distal end 252 and is juxtapositionally arranged with stapling jaw 254 to form an operational stapler head. In the deployment configuration the anvil jaw 253 may oppose and/or overlap with stapling jaw 254.

Stapling jaw 254 is optionally coupled to elongated body distal end 252 and lying distally thereto in both delivery configuration and deployment configuration. Anvil jaw 253 is configured to revolve around a point 256 located thereon on shifting, as shown in FIG. 12B. Optionally, anvil jaw 253 is further configured to translate relative to stapling jaw 254 on shifting, optionally during or after revolving. Revolving may be actuated about a plurality of pivot points, such as points 256 and 256′, provided along anvil jaw 253 and/or along elongated body 251. Optionally, revolving is part of a planar translation motion based on a closed chain linkage motion, optionally ‘four-bar-linkage’ being its simplest form structure (i.e., constructed from four links connected in a loop by four one degree of freedom joints), including the followings as four linkages: anvil jaw 253, part of elongated body 251 and two opposing revolving bars 255. Optionally, the four-bar linkage is configured and dimensioned to act as a parallelogram linkage having two opposing linkages identical in size. Anvil jaw 253 may then interlock with elongated body distal end 252 and/or with stapling jaw 254 in the deployment configuration to form the operational stapler head distally adjacent elongated body distal end 252. Stapling jaw 254 and anvil jaw 253 are then movable with respect to each other from an open position, wherein the jaws are spaced apart, to a closed position, wherein the jaws are in close proximity to each other. The maximal stapler cross-sectional dimension in the deployment configuration is equal to or greater than minimal inner diameter, and may be a maximal height, a maximal width and/or a maximal diameter. The maximal stapler cross-sectional dimension in the deployment configuration is equal to or greater than about 3 mm, optionally, equal to or greater than about 5 mm, optionally, equal to or greater than about 8 mm, optionally, equal to or greater than about 12 mm, optionally, equal to or greater than about 20 mm, optionally, equal to or greater than about 30 mm, or larger, or smaller, or an intermediate value.

Linear tissue stapler 260 (FIG. 12C) includes an elongated body 261 with an elongated body distal end 262, an anvil jaw 263, and a stapling jaw 264 containing parallel rows of tissue fasteners. Linear tissue stapler 260 is convertible from a delivery configuration (e.g., similar to that shown in FIG. 11A) to a deployment configuration (e.g., similar to that shown in FIG. 11B). When in the delivery configuration, linear tissue stapler 260 is sized to pass through a passage enclosing a minimal inner diameter, such a lumen enclosed by a laparoscopic sheath, port or trocar, or an endoscopic channel. The minimal inner diameter may be equal to or less than about 20 mm, optionally, equal to or less than about 12 mm, optionally, equal to or less than about 8 mm, optionally, equal to or less than about 6 mm, optionally, equal to or less than about 4 mm, or larger, or smaller, or an intermediate size. Anvil jaw 263 and stapling jaw 264 may be consecutively arranged lengthwise whereby their maximal cross-sectional dimension is smaller than minimal inner diameter. When in the delivery configuration, anvil jaw 263 may be positioned entirely proximally to elongated body distal end 262 making stapler 260 absent of an operational stapler head. When in the deployment configuration, anvil jaw 263 is positioned mostly or entirely distally to elongated body distal end 262 and is juxtapositionally arranged with stapling jaw 264 to form an operational stapler head. In the deployment configuration the anvil jaw 263 may oppose and/or overlap with stapling jaw 264.

Stapling jaw 264 is optionally coupled to elongated body distal end 262 and lying distally thereto in both delivery configuration and deployment configuration. Anvil jaw 263 is configured to slide proximally and/or distally when shifting between the delivery configuration and the deployment configuration, for example along a track 265 provided thereon or along part of elongated body 261. Anvil jaw 263 may then interlock with elongated body distal end 262 and/or with stapling jaw 264 in the deployment configuration to form the operational stapler head distally adjacent elongated body distal end 262. Stapling jaw 264 and anvil jaw 263 are then movable with respect to each other from an open position, wherein the jaws are spaced apart, to a closed position, wherein the jaws are in close proximity to each other. The maximal stapler cross-sectional dimension in the deployment configuration is equal to or greater than minimal inner diameter, and may be a maximal height, a maximal width and/or a maximal diameter. The maximal stapler cross-sectional dimension in the deployment configuration is equal to or greater than about 3 mm, optionally, equal to or greater than about 5 mm, optionally, equal to or greater than about 8 mm, optionally, equal to or greater than about 12 mm, optionally, equal to or greater than about 20 mm, optionally, equal to or greater than about 30 mm, or larger, or smaller, or an intermediate value.

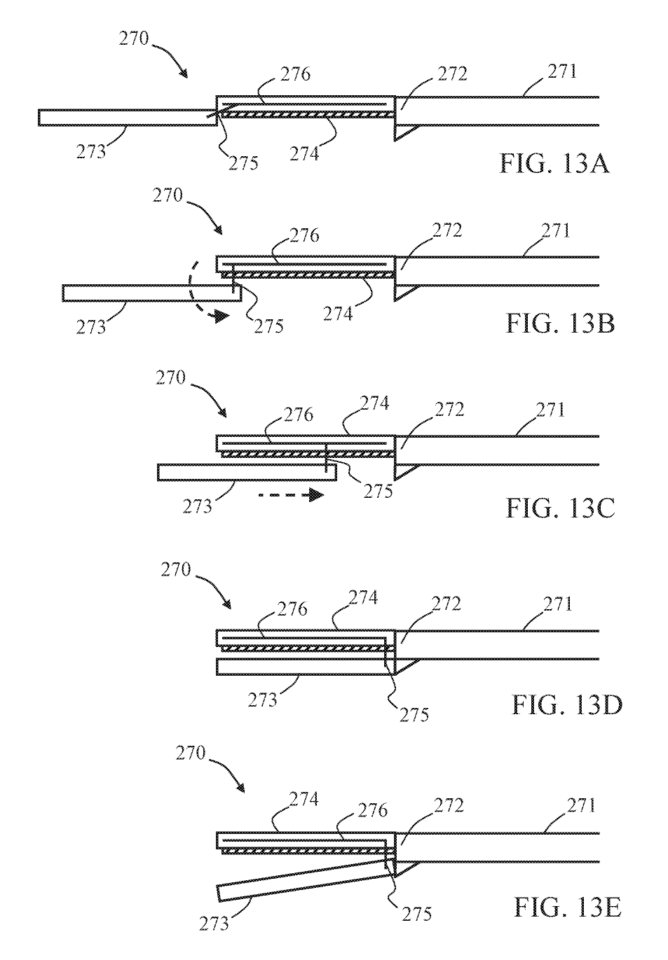

FIGS. 13A-13E schematically illustrate side views of exemplary embodiments of different positions in deploying an exemplary linear tissue stapler 270 configured to have its anvil jaw positioned distally to the stapling jaw when in the delivery configuration. Linear tissue stapler 270 includes an elongated body 271 with an elongated body distal end 272, an anvil jaw 273, and a stapling jaw 274 containing parallel rows of tissue fasteners. Linear tissue stapler 270 is convertible from a delivery configuration (FIG. 13A) to a deployment configuration (FIG. 13D). When in the delivery configuration, linear tissue stapler 270 is sized to pass through a passage enclosing a minimal inner diameter, such a lumen enclosed by a laparoscopic sheath, port or trocar, or an endoscopic channel. The minimal inner diameter may be equal to or less than about 20 mm, optionally, equal to or less than about 12 mm, optionally, equal to or less than about 8 mm, optionally, equal to or less than about 6 mm, optionally, equal to or less than about 4 mm, or larger, or smaller, or an intermediate size. Anvil jaw 273 and stapling jaw 274 may be consecutively arranged lengthwise whereby maximal cross-sectional dimension of stapler 270 is smaller than minimal inner diameter. When in the delivery configuration, anvil jaw 273 is positioned mostly or entirely distally to stapling jaw 274. When in the deployment configuration, anvil jaw 273 is juxtapositionally arranged with stapling jaw 274 to form an operational stapler head, optionally opposing and/or overlapping with stapling jaw 274.

Stapling jaw 274 is optionally coupled to elongated body distal end 272 and lying distally thereto in both delivery configuration and deployment configuration. Anvil jaw 273 is configured to swivel towards stapling jaw 274 (FIGS. 13A-13B) when shifting between the delivery configuration and the deployment configuration. Swiveling may be actuated about a pivot point provided at or adjacent distal end of stapling jaw 274, optionally facilitated by a hinge or a rotating arm 275. The shifting towards deployment configuration is optionally followed by proximal sliding motion of anvil jaw 273 (FIG. 13C), optionally along a slot or track 276, optionally provided along stapling jaw 274, optionally using rotating arm 275 coupled to track 276 with a runner (e.g., similar to that shown in FIG. 9A). Anvil jaw 273 may then interlock with elongated body distal end 272 and/or with stapling jaw 274 in the deployment configuration to form the operational stapler head distally adjacent elongated body distal end 272 (FIG. 13D). Stapling jaw 274 and anvil jaw 273, once forming an operational stapling head, are movable with respect to each other from an open position (FIG. 13E), wherein the jaws are spaced apart, to a closed position (FIG. 41), wherein the jaws are in close proximity to each other. The maximal stapler cross-sectional dimension in the deployment configuration is equal to or greater than minimal inner diameter, and may be a maximal height, a maximal width and/or a maximal diameter. The maximal stapler cross-sectional dimension in the deployment configuration is equal to or greater than about 3 mm, optionally, equal to or greater than about 5 mm, optionally, equal to or greater than about 8 mm, optionally, equal to or greater than about 12 mm, optionally, equal to or greater than about 20 mm, optionally, equal to or greater than about 30 mm, or larger, or smaller, or an intermediate value.

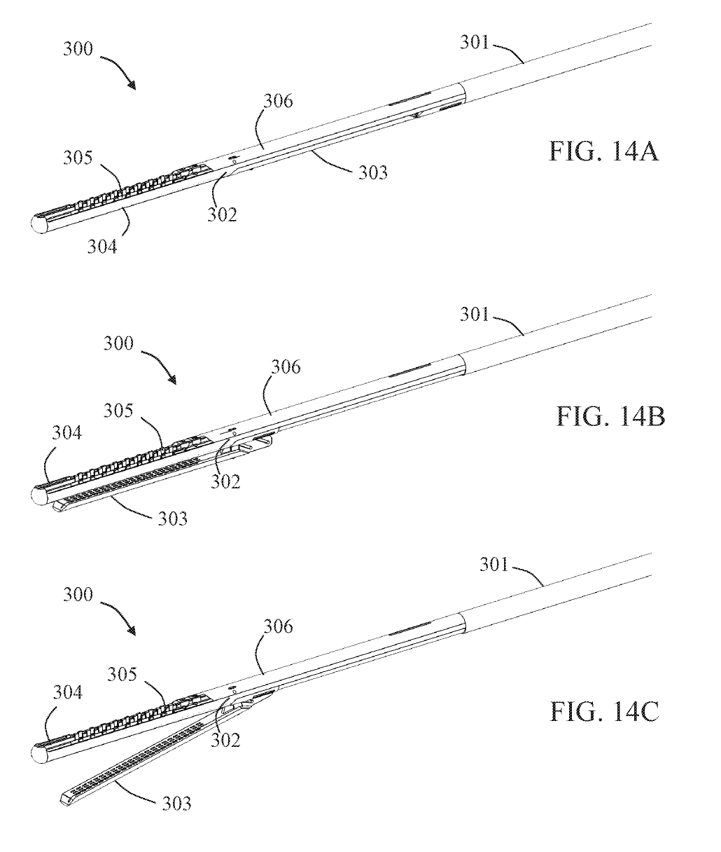

Reference is now made to FIGS. 14A-14C, which illustrate isometric views of exemplary embodiments of different positions in deploying an exemplary linear tissue stapler 300 configured to have its anvil jaw positioned proximally to the stapling jaw when in its delivery configuration. Linear tissue stapler 300 includes an elongated body 301 with an elongated body distal end 302, an anvil jaw 303, and a stapling jaw 304 containing parallel rows 305 of tissue fasteners. Linear tissue stapler 300 is convertible from a delivery configuration (FIG. 14A) to a deployment configuration (FIG. 14B). When in the delivery configuration, linear tissue stapler 300 is sized to pass through a passage enclosing a minimal inner diameter, such a lumen enclosed by a laparoscopic sheath, port or trocar, or an endoscopic channel. The minimal inner diameter may be equal to or less than about 20 mm, optionally, equal to or less than about 12 mm, optionally, equal to or less than about 8 mm, optionally, equal to or less than about 6 mm, optionally, equal to or less than about 4 mm, or larger, or smaller, or an intermediate size. Anvil jaw 303 and stapling jaw 304 are consecutively arranged lengthwise (anvil jaw 303 is positioned behind or proximal to stapling jaw 304) whereby their maximal cross-sectional dimension is smaller than minimal inner diameter. When in the delivery configuration, anvil jaw 303 is positioned entirely proximally to elongated body distal end 302 making stapler 300 absent of an operational stapler head. When in the deployment configuration, anvil jaw 303 is positioned mostly distally to elongated body distal end 302 and is juxtapositionally arranged partially overlapping with stapling jaw 304 to form an operational stapler head. A drive member 306 in the form of a slidable cover is shown in a fully retracted position distally to stapling jaw 104. Drive member 106 is provided with a blade 307 having a sharp edge 308 extending between the jaws.

Stapling jaw 304 is an extension of elongated body distal end 302, optionally coupled thereto or unitary with it, and lying distally thereto in both delivery configuration and deployment configuration. When shifting between the delivery configuration and the deployment configuration, anvil jaw 303 goes through a planar translation derived from a parallelogram linkage coupling mechanism and then undergoes a sliding motion until docking in a predetermined posture below stapling jaw 304. Anvil jaw 303 may then interlock with elongated body distal end 302 and/or with stapling jaw 304 to form an operational stapler head. Stapling jaw 304 and anvil jaw 303 are then movable with respect to each other from an open position (FIG. 14C), wherein the jaws are spaced apart, to a closed position (FIG. 14B), wherein the jaws are in close proximity to each other. The maximal stapler cross-sectional dimension in the deployment configuration may be a maximal height, a maximal width and/or a maximal diameter. The maximal stapler cross-sectional dimension in the deployment configuration is equal to or greater than about 3 mm, optionally, equal to or greater than about 5 mm, optionally, equal to or greater than about 8 mm, optionally, equal to or greater than about 12 mm, optionally, equal to or greater than about 20 mm, optionally, equal to or greater than about 30 mm, or larger, or smaller, or an intermediate value.

Each of the following terms written in singular grammatical form: ‘a’, ‘an’, and ‘the’, as used herein, means ‘at least one’, or ‘one or more’. Use of the phrase ‘one or more’ herein does not alter this intended meaning of ‘a’, ‘an’, or ‘the’. Accordingly, the terms ‘a’, ‘an’, and ‘the’, as used herein, may also refer to, and encompass, a plurality of the stated entity or object, unless otherwise specifically defined or stated herein, or, unless the context dearly dictates otherwise. For example, the phrases: ‘a unit’, ‘a device’, ‘an assembly’, ‘a mechanism’, ‘a component’, ‘an element’, and ‘a step or procedure’, as used herein, may also refer to, and encompass, a plurality of units, a plurality of devices, a plurality of assemblies, a plurality of mechanisms, a plurality of components, a plurality of elements, and, a plurality of steps or procedures, respectively.

Each of the following terms: ‘includes’, ‘including’, ‘has’. ‘having’. ‘comprises’, and ‘comprising’, and, their linguistic/grammatical variants, derivatives, or/and conjugates, as used herein, means ‘including, but not limited to’, and is to be taken as specifying the stated component(s), feature(s), characteristic(s), parameter(s), integer(s), or step(s), and does not preclude addition of one or more additional component(s), feature(s), characteristic(s), parameter(s), integer(s), step(s), or groups thereof. Each of these terms is considered equivalent in meaning to the phrase ‘consisting essentially of’.

Each of the phrases ‘consisting of’ and ‘consists of’, as used herein, means ‘including and limited to’.

The phrase ‘consisting essentially of’, as used herein, means that the stated entity or item (system, system unit, system sub-unit, device, assembly, sub-assembly, mechanism, structure, component, element or, peripheral equipment, utility, accessory, or material, method or process, step or procedure, sub-step or sub-procedure), which is an entirety or part of an exemplary embodiment of the disclosed invention, or/and which is used for implementing an exemplary embodiment of the disclosed invention, may include at least one additional ‘feature or characteristic’ being a system unit, system sub-unit, device, assembly, sub-assembly, mechanism, structure, component, or element, or, peripheral equipment utility, accessory, or material, step or procedure, sub-step or sub-procedure), but only if each such additional ‘feature or characteristic’ does not materially alter the basic novel and inventive characteristics or special technical features, of the claimed entity or item.

The term ‘method’, as used herein, refers to steps, procedures, manners, means, or/and techniques, for accomplishing a given task including, but not limited to, those steps, procedures, manners, means, or/and techniques, either known to, or readily developed from known steps, procedures, manners, means, or/and techniques, by practitioners in the relevant field(s) of the disclosed invention.

Throughout this disclosure, a numerical value of a parameter, feature, characteristic, object, or dimension, may be stated or described in terms of a numerical range format Such a numerical range format as used herein, illustrates implementation of some exemplary embodiments of the invention, and does not inflexibly limit the scope of the exemplary embodiments of the invention. Accordingly, a stated or described numerical range also refers to, and encompasses, all possible sub-ranges and individual numerical values (where a numerical value may be expressed as a whole, integral, or fractional number) within that stated or described numerical range. For example, a stated or described numerical range ‘from 1 to 6’ also refers to, and encompasses, all possible sub-ranges, such as ‘from 1 to 3’, ‘from 1 to 4’, ‘from 1 to 5’, ‘from 2 to 4’. ‘from 2 to 6’, ‘from 3 to 6’, etc., and individual numerical values, such as ‘1’, ‘1.3’, ‘2’, ‘2.8’, ‘3’, ‘3.5’, ‘4’, ‘4.6’, ‘5’, ‘5.2’, and ‘6’, within the stated or described numerical range of ‘from 1 to 6’. This applies regardless of the numerical breadth, extent, or size, of the stated or described numerical range.

Moreover, for stating or describing a numerical range, the phrase ‘in a range of between about a first numerical value and about a second numerical value’, is considered equivalent to, and meaning the same as, the phrase ‘in a range of from about a first numerical value to about a second numerical value’, and, thus, the two equivalently meaning phrases may be used interchangeably. For example, for stating or describing the numerical range of room temperature, the phrase ‘room temperature refers to a temperature in a range of between about 20° C. and about 25° C.’, and is considered equivalent to, and meaning the same as, the phrase ‘room temperature refers to a temperature in a range of from about 20° C. to about 25° C.’.

The term ‘about’, as used herein, refers to +10% of the stated numerical value.

It is to be fully understood that certain aspects, characteristics, and features, of the invention, which are, for clarity, illustratively described and presented in the context or format of a plurality of separate embodiments, may also be illustratively described and presented in any suitable combination or sub-combination in the context or format of a single embodiment. Conversely, various aspects, characteristics, and features, of the invention which are illustratively described and presented in combination or sub-combination in the context or format of a single embodiment, may also be illustratively described and presented in the context or format of a plurality of separate embodiments.

Although the invention has been illustratively described and presented by way of specific exemplary embodiments, and examples thereof, it is evident that many alternatives, modifications, or/and variations, thereof, will be apparent to those skilled in the art. Accordingly, it is intended that all such alternatives, modifications, or/and variations, fall within the spirit of, and are encompassed by, the broad scope of the appended claims.

All publications, patents, and or/and patent applications, cited or referred to in this disclosure are herein incorporated in their entirety by reference into the specification, to the same extent as if each individual publication, patent, or/and patent application, was specifically and individually indicated to be incorporated herein by reference. In addition, citation or identification of any reference in this specification shall not be construed or understood as an admission that such reference represents or corresponds to prior art of the present invention. To the extent that section headings are used, they should not be construed as necessarily to limiting.

1. A linear tissue stapler, comprising:
an elongated body having a longitudinal axis; and
a stapling head, including at least a first member and a second member, at least one of said first member and said second member includes at least one row of tissue fasteners;
wherein the linear tissue stapler is convertible from a delivery configuration, whereby said elongated body and said stapling head are passable through a passage enclosing a minimal inner diameter, to a deployment configuration, whereby said stapling head is operational;
wherein, in said delivery configuration, said first member and said second member are consecutively arranged lengthwise relative to said longitudinal axis, such that an overall diameter thereof is smaller than said minimal inner diameter, and, in said deployment configuration, said first member and said second member are juxtapositionally arranged relative to said longitudinal axis, such that an overall diameter thereof is equal to or greater than said minimal inner diameter.

2. The linear tissue stapler of claim 1, comprising a first track, wherein, in said delivery configuration, said first member contacts said first track at a first location, and, in said deployment configuration, said first member contacts said first track at a second location.

3.-5. (canceled)

6. The linear tissue stapler of claim 2, wherein, upon converting from said delivery configuration to said deployment configuration, said first track is configured such that said first member travels lengthwise or/and rotates relative to said longitudinal axis.

7.-9. (canceled)

10. The linear tissue stapler of claim 1, wherein at least one of said first member and said second member includes at least three of said rows of said tissue fasteners.

11. The linear tissue stapler of claim 1, wherein each of said at least one row of said tissue fasteners includes at least five of said tissue fasteners.

12.-14. (canceled)

15. The linear tissue stapler of claim 1, wherein said second member is an anvil jaw, and said first member is a stapling jaw including a plurality of said rows of tissue fasteners, said rows being parallel to each other.

16. The linear tissue stapler of claim 15, wherein said stapling head includes a stapling head actuating assembly, and wherein said anvil jaw is pivotally connected to said elongated body and is operable with said stapling head actuating assembly.

17.-20. (canceled)

21. The linear tissue stapler of claim 1, further comprising a driving member including a number of runners, each of said runners is slidable along a corresponding grooved route extending along a length of one of said first and second members.

22.-31. (canceled)

32. A linear tissue stapler of claim 1, wherein said first member transversally shifts between said delivery configuration and said deployment configuration, to a position juxtaposed to said second member.

33. A method of surgically fastening or/and cutting tissue inside a body, the method comprising:
providing a passage connecting between environment outside of the body and a location inside the body in a vicinity of the tissue;
passing a linear tissue stapler through said passage towards said inside body location, said linear tissue stapler comprises an elongated body having a longitudinal axis, and, a stapling head including a first member and a second member arranged in a delivery configuration, in which said first and second members are consecutively arranged lengthwise relative to said longitudinal axis;
effecting emergence of said first member and said second member out of said passage;
converting said linear tissue stapler from said delivery configuration to a deployment configuration, in which said first member and said second member are juxtapositionally arranged relative to said longitudinal axis and interlocked with each other to rigidly form an operational stapling head comprising a stapling jaw and an anvil jaw;
clasping the tissue between said stapling jaw and said anvil jaw; and
actuating said stapling head to release a plurality of tissue fasteners from said stapling jaw through the tissue towards said anvil jaw.

34. The method of claim 33, further comprising providing a blade in a spaced passage extending along said stapling jaw of said stapling head, wherein said actuating includes, or is followed by, sliding said blade along said spaced passage so as to cut through said clasped tissue with a sharp edge of said blade.

35. The method of claim 33, wherein said passage encloses a minimal inner diameter equal to or less than about 6 mm, wherein said stapling jaw, in said deployment configuration, has overall diameter greater than said minimal inner diameter.

36.-47. (canceled)

48. A linear tissue stapler, comprising:
a stapling jaw, including parallel rows of tissue fasteners; and
an anvil jaw;
wherein the linear tissue stapler is convertible from a delivery configuration, whereby said stapling jaw and said anvil jaw are passable through a passage enclosing a minimal inner diameter, to a deployment configuration including an operational stapling head;
wherein, in said delivery configuration, said stapling jaw and said anvil jaw are consecutively arranged lengthwise such that a maximal stapler cross-sectional dimension is smaller than said minimal inner diameter, and in said deployment configuration, said stapling jaw and said anvil jaw are juxtapositionally arranged and form said operational stapling head, whereby said maximal stapler cross-sectional dimension is equal to or greater than said minimal inner diameter.

49. The linear tissue stapler of claim 48, further comprising an elongated body including an elongated body distal end.

50. The linear tissue stapler of claim 49, wherein, in said delivery configuration, said anvil jaw is at least partly positioned proximally to said elongated body distal end, and in said deployment configuration, said anvil jaw is at least partly positioned distally to said elongated body distal end.

51. The linear tissue stapler of claim 49, wherein said stapling jaw is coupled to said elongated body distal end and lying distally thereto in both of said delivery configuration and said deployment configuration.

52.-55. (canceled)

56. The linear tissue stapler of claim 48, wherein, in said deployment configuration, said anvil jaw overlaps said stapling jaw.

57. The linear tissue stapler of claim 48, wherein, when in said deployment configuration, and when forming said operational stapling head, said stapling jaw and said anvil jaw are movable with respect to each other from an open position, in which said jaws are spaced apart, to a closed position, in which said jaws are in close proximity to each other.

58.-59. (canceled)

60. The linear tissue stapler of claim 48, wherein said anvil jaw is configured to revolve around a point located thereon.

61. The linear tissue stapler of claim 48, wherein said anvil jaw is configured to translate relative to said stapling jaw.

62.-66. (canceled)
